(19) United States
(12) Patent Application Publication
(10) Pub. No.: US 2015/0353617 A1
(43) Pub. Date: Dec. 10, 2015

(54) ANTI-ANGIOGENIC VEGF-AX ISOFORM

(71) Applicant: THE CLEVELAND CLINIC FOUNDATION, Cleveland, OH (US)

(72) Inventors: Paul L. Fox, Shaker Heights, OH (US); Sandeepa M. Eswarappa, Cleveland, OH (US)

(21) Appl. No.: 14/763,261

(22) PCT Filed: Jan. 27, 2014
(86) PCT No.: PCT/US14/13109

§ 371 Date: Jul. 24, 2015

Related U.S. Application Data
(60) Provisional application No. 61/757,151, filed on Jan. 27, 2013.

(51) Int. Cl. C07K 14/515; G01N 33/74; C07K 16/22

(52) U.S. Cl. CPC C07K 14/515; C07K 16/22; G01N 33/74; C07K 2317/54; C07K 2317/55; C07K 2317/622; A61K 38/00

(57) ABSTRACT

A VEGF-Ax polypeptide, as well as a smaller Ax poly-peptide, both having anti-angiogenic activity that can be used for treating or preventing angiogenesis are described. An antibody specific to the Ax region of a VEGF-Ax polypeptide, as well as an antibody specific to the C-terminus region of a VEGF-A polypeptide that is not capable of binding to VEGF-Ax, and methods of using these antibodies, are also described.

### CONTINUING APPLICATION DATA

This application claims the benefit of U.S. Provisional Application Ser. No. 61/757,151 filed Jan. 27, 2013, which is incorporated by reference herein.

### GOVERNMENT FUNDING

This work was supported, at least in part, by grant numbers P01 HL029582, P01 HL076491, R01 GM086430, and R21 HL094841 from the Department of Health and Human Services, National Institutes of Health. The United States government has certain rights in this invention.

### SEQUENCE LISTING

The instant application contains a Sequence Listing which has been submitted electronically in ASCII format and is hereby incorporated by reference in its entirety. Said ASCII copy, created on Jan. 24, 2014, is named CCF-022000WOORD_SL.txt and is 19,990 bytes in size.

### BACKGROUND

Vascular endothelial growth factor-A (VEGF-A) is a member of the platelet derived growth factor/vascular endothelial growth factor family, and induces proliferation and migration in cultured endothelial cells (ECs). VEGF-A is also known to induce angiogenesis, vasculogenesis, promote cell migration, and inhibit apoptosis. It is central to angiogenesis and vasculogenesis during development, and pathological conditions including tumorigenesis. ECs not only respond to VEGF-A, they can also generate it, and EC-derived VEGF-A exhibits an intracellular activity essential for vascular homeostasis. Lee et al., Cell 130, 691-703 (2007).

Generation of anti-angiogenic VEGF-A isoforms, termed VEGF-Ab, by alternative splicing of VEGFA mRNA has been described. S. J. Harper, D. 0. Bates, Nat. Rev. Cancer 8, 880-887 (2008); Bates et al., Cancer Res. 62, 4123-4131 (2002). The proposed isoforms differ only in their six C-terminus amino acids: pro-angiogenic isoforms terminate with CDKPRR (SEQ ID NO: 10), while anti-angiogenic isoforms terminate with SLTRKD (SEQ ID NO: 11).

Angiogenesis is an important biological process, necessary for reproduction, development, and wound repair. Angiogenesis begins with the degradation of the basement membrane by proteases secreted from activated endothelial cells. In adults, the rate of proliferation of endothelial cells is generally low, and tissues are normally in a state of angiogenesis equilibrium in which growth factors that simulate new vessel growth are balanced by other factors which inhibit vessel growth. However, rapid proliferation of endothelial cells can occur during certain processes such as reproduction and wound healing. The rate of angiogenesis responds to a change in the levels of angiogenic growth factors.

Abnormal angiogenesis can occur in various diseases and disorders. For example, in ulcers, ischemic heart disease, and peripheral artery disease, insufficient levels of angiogenesis can contribute to the pathology of those conditions, while excessive angiogenesis is known to be involved in cancer, atherosclerosis, blindness, psoriasis, and rheumatoid arthritis. It is therefore desirable to be able to modulate the rate of angiogenesis in order to help prevent or treat these types of conditions, either by increasing or decreasing the rate of angiogenesis. Because various VEGF-A isoforms have been shown to have both angiogenic and antiangiogenic activity, these proteins and antibodies against these proteins such as the anti-VEGF-antibody “Bevacizumab” have been proposed or approved for therapeutic use. However, there remains a need for additional VEGF-A isoforms and antibodies against these isoforms, particularly ones that provide increased effectiveness for regulating angiogenesis.

### SUMMARY

Translational readthrough, observed primarily in less complex organisms from viruses to Drosophila, increases the protein repertoire by translation of select transcripts beyond the canonical stop codon. The inventors have shown that mammalian vascular endothelial growth factor-A (VEGFA) mRNA undergoes programmed translational readthrough (PTR) generating a novel anti-angiogenic VEGF-A isoform, VEGF-Ax. A cis-acting, 63-nt element in the VEGFA 3′UTR directs the PTR by decoding the UGA stop codon as serine. Heterogeneous nuclear ribonucleoprotein (hnRNP) A2/B 1 binds this element and promotes readthrough. Pathophysiological significance of VEGF-Ax is indicated by its expression in multiple tissues and diminution in adenocarcinoma of colon. Furthermore, genome-wide analysis and experimental validation revealed AGO1 and MTCH2 as authentic mammalian readthrough targets. Overall, the studies reveal a protein-regulated PTR event unprecedented in vertebrate mRNAs.

In view of this work, one aspect of the invention provides an isolated VEGF-Ax polypeptide having anti-angiogenic activity. The VEGF-Ax polypeptide includes a region having at least 85% identity to SEQ ID NO: 1 and having an amino terminus comprising a region having at least 85% identity to SEQ ID NO: 2. In another aspect, the invention provides a method of treating or preventing angiogenesis by administering to a subject a therapeutically effective amount of a VEGF-Ax polypeptide including a region having at least 85% identity to SEQ ID NO: 1 and having an amino terminus comprising a region having at least 85% identity to SEQ ID NO: 2.

Another aspect of the invention provides an isolated Ax polypeptide having anti-angiogenic activity, the polypeptide having from 22 to 50 amino acids and including a region having at least 85% identity to SEQ ID NO: 2. A further aspect of the invention provides a method of decreasing angiogenesis by administering to a subject a therapeutically effective amount of an Ax polypeptide having from 22 to 50 amino acids and including a region having at least 85% identity to SEQ ID NO: 2.

The invention also provides various antibodies specific for all or a portion of the VEGF-Ax polypeptide or the Ax polypeptide, and methods of using these antibodies. One aspect of the invention provides an antibody specific to the Ax region of a VEGF-Ax polypeptide capable of binding to a VEGF-Ax and not capable of binding to VEGF-A. These antibodies can be used in a method of prognosis of a disease or disorder associated with increased or abnormal angiogenesis in a subject that includes the steps of determining the level of VEGF-Ax in a biological sample from the subject by an immunoassay using an antibody specific to the Ax region of a VEGF-Ax polypeptide that is capable of binding to a VEGF-Ax and not capable of binding to VEGF-A; comparing the level of VEGF-Ax to a predetermined value based on levels of VEGF-Ax in comparable biological samples obtained from one or more control subjects; and providing a prognosis of increased severity of the disease or disorder for the subject if the level of VEGF-Ax in the subject is lower than the predetermined value. These antibodies can also be used in a method of stimulating angiogenesis in a subject that includes administering a therapeutically effective amount of an antibody specific to the Ax region of a VEGF-Ax polypeptide that is capable of binding to VEGF-Ax and not capable of binding to VEGF-A.

Another aspect of the invention provides an antibody specific to the C-terminus region of a VEGF-A polypeptide that is capable of binding to the C-terminus region of a VEGF-A polypeptide and not capable of binding to VEGF-Ax. These antibodies can be used in a method of decreasing angiogenesis in a subject that includes administering a therapeutically effective amount of an antibody specific to the C-terminus region of a VEGF-A polypeptide that is capable of binding to the C-terminus region of a VEGF-A polypeptide and not capable of binding to VEGF-Ax.

### BRIEF DESCRIPTION OF THE FIGURES

FIG. 1 provides graphs showing the activity of VEGF-Ax. (A) shows that VEGF-Ax (25 ng/ml) has higher anti-angiogenic activity in comparison with VEGF-Ab (250 ng/ml) when tested for effect on primary EC proliferation in a serum-free medium, while (B) shows that VEGF-Ax both suppresses VEGF-A angiogenic activity, and exhibits potent anti-angiogenic activity when used alone in the same assay, with increasing amounts of VEGF-Ax showing increasing activity.

FIG. 2 provides a schematic representation that compares the generation of VEGF-A, VEGF-Ab, and VEGF-Ax from mRNA, as well as the resultant proteins. VEGF-A is formed by translation of the open reading frame, ending in the amino acid sequence CDKPRR (SEQ ID NO: 10) to provide the conventional pro-angiogenic form. VEGF-Ab, on the other hand, is generated by alternate splicing at the C-terminus, providing an anti-angiogenic protein ending in the amino acid sequence SLTRKD (SEQ ID NO: 11). Finally, VEGF-Ax is formed by a translational readthrough, to provide a sequence that includes the CDKPRR (SEQ ID NO: 10) sequence, but also includes the 22-amino acid Ax peptide extension and ends in the amino acid sequence SLTRKD (SEQ ID NO: 11). FIG. 2 discloses SEQ ID NOS 10-11, respectively, in order of appearance.

FIG. 3 provides a schematic representation of the VEGF-A gene, and various VEGF-A protein isoforms that can result of the expression of different exons within that gene.

FIG. 4 provides the amino acid sequences for VEGF-A111x, VEGF-A121x, VEGF-A148x, VEGF-A165x, VEGF-A183x, and VEGF-A189x.

FIG. 5 provides a graph showing the anti-angiogenic activity of the Ax polypeptide alone and when used together with angiogenic VEGF-A, when tested for effect on primary EC proliferation in a serum-free medium.

FIG. 6 provides a graph showing the antitumor effectiveness of VEGF-Ax. Nu/Nu athymic nude mice were inoculated with HCT116 human colon carcinoma cells. Mice were injected subcutaneously on both flanks with VEGF-Ax-enriched fraction of conditioned medium obtained from HEK293 cells transfected with plasmid expressing bovine VEGF-Ax^{Ser} cDNA. VEGF-Ax^{Ser} cDNA is identical to VEGF-Ax cDNA except that the UGA stop codon is replaced by a Ser codon to improve efficiency of translation. Conditioned medium was enriched by binding to heparin column followed by elution with high salt buffer. The control group was injected with elution buffer. VEGF-Ax injection was given every 2 to 3 days starting from 4th day of tumor innoculation. Tumor progression was monitored by measuring its volume with calipers.

FIG. 7 provides graphs and images of data obtained from endothelial cells (ECs) that express a novel VEGF-A isoform, VEGF-Ax. (A) Neutralizing anti-VEGF-A antibody enhances EC migration. Shown are ECs migrating across razor-wound line (mean±SE, n=3) (left), individual cell trajectories and root-mean-square displacement (center), and cell speed in the presence of anti-VEGF-A (n=71) and IgG (n=52) (right). (B) Neutralizing anti-VEGF-A antibody increases EC proliferation (mean ±SE, n=4). (C) Conservation of VEGFA 3′UTR proximal region and downstream, in-frame stop codon in mammals (SEQ ID NOS 29-40, respectively, in order of appearance). (D) Schematic of VEGF-A, -Ab, and -Ax generation. FIG. 7(D) discloses SEQ ID NOS 10-11, respectively, in order of appearance. (E) Immunoblot showing VEGF-Ax expression in ECs transfected with three different VEGFA-specific siRNAs. (F) EC lysate was immunoprecipitated with anti-VEGF-Ax antibody and subjected to immunoblot analysis. (G) HEK293 cells were transfected with Myc-tagged VEGFA cDNA in which the canonical stop codon is mutated to GCA (VEGF-Ax^{Ala}), or with Myc-tagged VEGFA cDNA, and conditioned media subjected to immunoblot analysis. (H) Anti-VEGF-A antibody detects VEGF-A (arrowhead) and a higher molecular weight isoform (arrow) consistent with VEGF-Ax. EC lysates were deglycosylated and resolved on 16% Tricine gel. (I) Immunoblot analysis of lysates from murine aortic ECs (MAEC), bovine aortic ECs (BAEC), and human umbilical vein ECs (HUVEC).

FIG. 8 provides graphs and sequence representations showing that the Ax element is necessary and sufficient for translational readthrough. (A) Plasmids containing in-frame VEGF-Ax-FLuc (firefly luciferase), and variants with TGA-to-GCA substitution, no Ax element, Ax replaced by a non-specific sequence (ns), and two in-frame stop codons downstream of the canonical stop codon (TGA-TAA-TAA) were transfected into ECs. FLuc activity was normalized by expression of co-transfected Renilla Luc (top) and FLuc mRNA expression determined by qRT-PCR (bottom). (B) Chimeric plasmids containing Myc-Ax-FLuc and its variants were transfected into ECs. Relative FLuc activity and mRNA expression are shown (left). Readthrough product was detected by immunoblot following immunoprecipitation with anti-Myc-tag antibody (top-right). Plasmids were subjected to coupled transcription-translation in vitro using wheat germ extract and FLuc activity determined (bottom-right). (C) Nucleotide sequence (SEQ ID NO: 41) of bovine Ax element with flanking stop codons (boxes). Deletions are illustrated pictorially and mutations indicated by arrows. (D,E) Plasmids containing deletions (D) or mutations (E) of VEGF-Ax-FLuc were transfected into ECs. FLuc activity and normalized by expression of co-transfected Renilla Luc (top); FLuc mRNA expression was determined by qRT-PCR (bottom). Error bars represent mean±SE (n=3); P-values and readthrough efficiencies (in %) are indicated.

FIG. 9 provides graphs and images showing that hnRNP A2/B1 facilitates VEGFA mRNA translation readthrough (A) Human VEGFA inter-stop codon sequence (SEQ ID NO: 42) (Ax element) contains a near-consensus hnRNP A2/B1 response element (A2RE) (SEQ ID NO: 43). (B) Surface plasmon resonance analysis of recombinant hnRNP A2/B1 binding to biotinylated RNA containing VEGFA A2RE immobilized on streptavidin sensor chip (in response units, RU). (C) Cytoplasmic localization of hnRNP A2/B1 in ECs shown by immunofluorescence and immunoblot. (D) Immunoprecipitation of hnRNP A2/B1 followed by RT-PCR using VEGFA- and GAPDH-specific primers. (E) VEGF-Ax-FLuc plasmid containing an Ax element with A2RE mutation (mut. Ax) was transfected into ECs. Anti-hnRNP A2/B1 immunoprecipitates were probed by qRT-PCR using FLuc- and GAPDH-specific TaqMan probes (top). Readthrough expressed as relative FLuc activity and mRNA level are shown (bottom 2 panels) (mean ±SE, n=3). FIG. 9(E) discloses SEQ ID NOS 44-45, respectively, in order of appearance. (F) Following siRNA-mediated knockdown of hnRNP A2/B1, ECs were transfected with VEGF-Ax-FLuc construct and its variants; FLuc activity and mRNA determined by qRT-PCR are shown (top 2 panels). VEGF-Ax and VEGF-A were determined by immunoblot analysis and VEGFA mRNA by qRT-PCR (bottom 5 panels).

FIG. 10 provides graphs and images showing the physiological significance of translational readthrough. (A) Immunoblot of VEGF-Ax in EC-conditioned medium not supplemented with serum or growth factors. (B) EC migration and (C) proliferation (*, P<0.05, 2-way ANOVA) in presence of anti-VEGF-Ax antibody or recombinant human VEGF-A (20 ng/ml) (mean±SE, n=3). (D) EC migration (n=3), (E) tube formation in Matrigel (n=7), and (F) proliferation in the presence of purified, recombinant His-VEGF-Ax^{ala} (50 ng/ml) (n=3). (G) EC migration following hnRNP A2/B 1 knockdown. (H) Immunofluorescence of normal and Grade 2 adenocarcinoma of colon. VEGF-Ax and total VEGF-A were quantified as integrated, background-subtracted fluorescence intensity; expression in Grade 1 (n=6) and Grade 2/3 colon adenocarcinoma (n=22) were normalized to healthy colon (n=5); whiskers show 5^{th} and 95^{th} percentiles. (I) Candidate readthrough mRNAs were selected by genome-wide, bioinformatic screen. About 700 nt at the 3′ terminus of human coding sequences were cloned upstream of the inter-stop codon region (ISR) and in-frame with FLuc, and transfected into HEK293 cells. Relative FLuc activities normalized to FLuc mRNA (determined by qRT-PCR) are shown (mean±SE, n=3).

### DETAILED DESCRIPTION

Unless otherwise defined, all technical and scientific terms used herein have the same meaning as commonly understood by one of ordinary skill in the art to which this application pertains. Although methods and materials similar or equivalent to those described herein can be used in the practice or testing of the exemplary embodiments, suitable methods and materials are described below. In case of conflict, the present specification, including definitions, will control. In addition, the materials, methods, and examples are illustrative only and not intended to be limiting.

The terminology as set forth herein is for description of the embodiments only and should not be construed as limiting the application as a whole. Unless otherwise specified, “a,” “an,” “the,” and “at least one” are used interchangeably. Furthermore, as used in the description of the application and the appended claims, the singular forms “a”, “an”, and “the” are inclusive of their plural forms, unless contraindicated by the context surrounding such. Furthermore, the recitation of numerical ranges by endpoints includes all of the numbers subsumed within that range (e.g., 1 to 5 includes 1, 1.5, 2, 2.75, 3, 3.80, 4, 5, etc.).

The term “isolated” means that the material is removed from its original environment (e.g., the natural environment if it is naturally occurring) or is synthetically derived. For example, a naturally-occurring polypeptide present in a living animal is not isolated, but the same polypeptide, separated from some or all of the coexisting materials in the natural system, is isolated. Such a polypeptide could be part of a composition, and still be isolated in the composition, and not be a part of its natural environment.

As used herein, the term “polypeptide” refers to an oligopeptide, peptide, or protein sequence, or to a fragment, portion, or subunit of any of these, and to naturally occurring or synthetic molecules. The term “polypeptide” also includes amino acids joined to each other by peptide bonds or modified peptide bonds, i.e., peptide isosteres, and may contain any type of modified amino acids. The term “polypeptide” also includes peptides and polypeptide fragments, motifs and the like, glycosylated polypeptides, all “mimetic” and “peptidomimetic” polypeptide forms, and retro-inversion peptides (also referred to as all-D-retro or retro-enantio peptides).

As used herein, the term “antibody” refers to whole antibodies, e.g., of any isotype (IgG, IgA, IgM, IgE, etc.) and includes fragments thereof, which are also specifically reactive with a target polypeptide. Antibodies can be fragmented using conventional techniques and the fragments screened for utility and/or interaction with a specific epitope of interest. Thus, the term includes segments of proteolytically-cleaved or recombinantly-prepared portions of an antibody molecule that are capable of selectively reacting with a certain polypeptide, such as single-chain antibodies (SAb). Non-limiting examples of such proteolytic and/or recombinant fragments include Fab, F(ab′)₂, Fab′, Fv, and SAb containing a V[L] and/or V[H] domain joined by a peptide linker. The SAb' s may be covalently or non-covalently linked to form antibodies having two or more binding sites. The term “antibody” also includes polyclonal, monoclonal, nanobodies, or other purified preparations of antibodies, recombinant antibodies, monovalent antibodies, and multivalent antibodies. Antibodies may be humanized and may further include engineered complexes that comprise antibody-derived binding sites, such as diabodies and triabodies.

The term monoclonal antibody, as used herein, refers to antibodies that are identical because they are produced by one type of immune cell that are all clones of a single parent cell. The monoclonal antibodies of the present invention can include intact monoclonal antibodies, antibody fragments, conjugates, or fusion proteins, which contain a V_{H}-V_{L} pair where the complementarity determining region forms the antigen binding site.

The term chimeric antibody, as used herein, refers to an antibody which includes sequences derived from two different antibodies, which typically are of different species. Most typically, chimeric antibodies include human and non-human antibody fragments, generally human constant and non-human variable regions.

The term humanized antibody, as used herein, refers to an antibody derived from a non-human antibody, and a human antibody which retains or substantially retains the antigen-binding properties of the parent antibody but which is less immunogenic in humans.

The term antigen, as used herein, refers to a molecule or a portion of a molecule capable of being bound by an antibody which is additionally capable of inducing an animal to produce an antibody capable of binding to an epitope of that antigen. An antigen can have one or more than one epitope. The specific reaction referred to above is meant to indicate that the antigen will react, in a highly selective manner, with its corresponding antibody and not with the multitude of other antibodies which can be evoked by other antigens.

The term epitope, as used herein, refers to that portion of any molecule capable of being recognized by, and bound by, an antibody. In general, epitopes consist of chemically active surface groupings of molecules, for example, amino acids or sugar side chains, and have specific three-dimensional structural characteristics as well as specific charge characteristics. The epitopes of interest for the present invention are epitopes comprising amino acids.

As used herein, the terms “treatment,” “treating,” or “treat” refer to any treatment of an angiogenesis-mediated disease in a subject including, but not limited to, inhibiting disease development, arresting development of clinical symptoms associated with the disease, relieving the symptoms associated with the disease, increasing the quality of life, and/or life expectancy of a subject. Prophylactic treatment or prevention refers to preventing the disease from developing. However, the terms “treating” and “ameliorating” do not necessarily indicate a reversal or cessation of the disease process underlying the disease or disorder afflicting the subject being treated. Such terms indicate that the deleterious signs and/or symptoms associated with the condition being treated are lessened or reduced, or the rate of progression is reduced, compared to that which would occur in the absence of treatment. A change in a disease sign or symptom can be assessed at the level of the subject (e.g., the function or condition of the subject is assessed), or at a tissue or cellular level (e.g., the production of markers associated with the disease are lessened or reduced).

As used herein, the term “diagnosis” can encompass determining the presence and nature of disease or condition in a subject. “Diagnosis” can also encompass diagnosis in the context of rational therapy, in which the diagnosis guides therapy, including initial selection of therapy, modification of therapy (e.g., adjustment of dose and/or dosage regimen), and the like. Diagnosis does not imply certainty with regard to the nature of the disease or condition identified, but rather the substantial likelihood that the disease or condition is present. For example, a subject diagnosed as having a disease or disorder associated with increased angiogenesis may be 2×, 10×, or 100× more likely to have the disease or disorder relative to a subject that has not been diagnosed as having the disease or disorder.

As used herein, the term “prognosis” refers to a prediction of the probable course and outcome of a disease, or the likelihood of recovery from a disease. Prognosis is distinguished from diagnosis in that it is generally already known that the subject has the disease, although prognosis and diagnosis can be carried out simultaneously, and by the fact that prognosis characterizes the type and severity of a disease rather than merely identifying its presence.

The terms “individual,” “host,” “subject,” and “patient” are used interchangeably herein, refers to a species of mammal, including, but not limited to, primates, including simians and humans, equines (e.g., horses), canines (e.g., dogs), felines, various domesticated livestock (e.g., ungulates, such as swine, pigs, goats, sheep, and the like), as well as domesticated pets and animals maintained in zoos.

The term “therapeutically effective amount” refers to an amount of polypeptide or antibody sufficient to exhibit a detectable therapeutic effect. The therapeutic effect may include, for example, without limitation, inhibiting the growth of undesired tissue or malignant cells, inhibiting inappropriate angiogenesis (neovascularization), limiting tissue damage caused by chronic inflammation, inhibition of tumor cell growth, and the like. The precise effective amount and time course of treatment for a subject will depend upon the subject's size and health, the nature and severity of the condition to be treated, and the like. Thus, it is not possible to specify an exact effective amount or time course in advance. However, the effective amount for a given situation can be determined by routine experimentation based on the information provided herein.

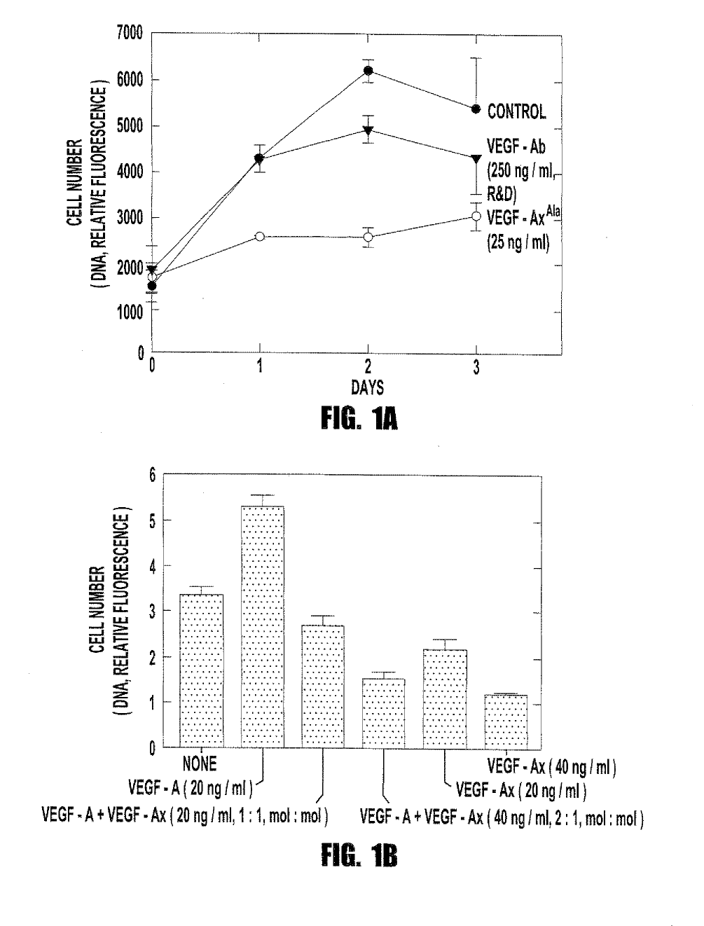

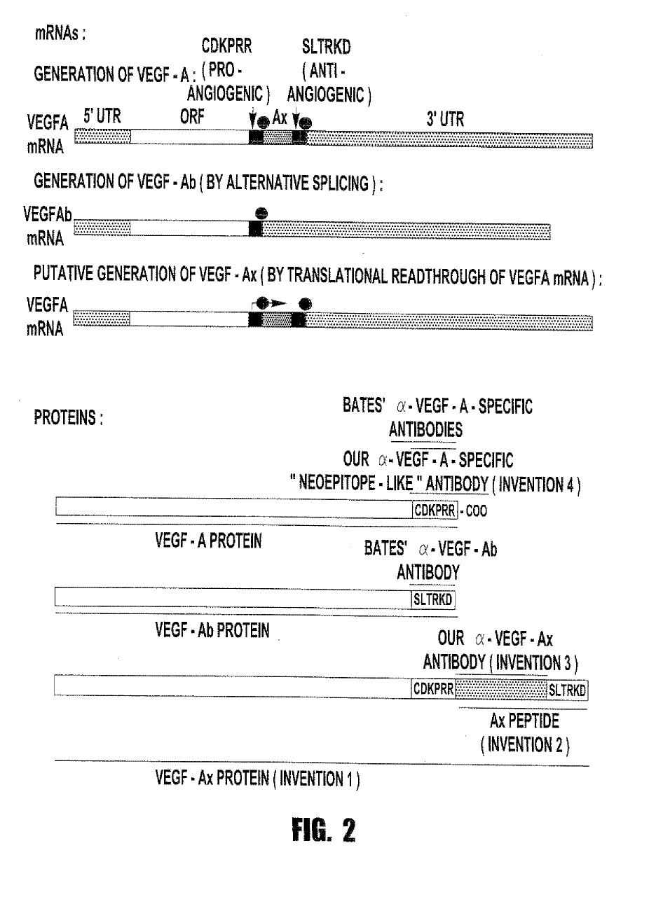

In one aspect, the present invention provides an isolated VEGF-Ax polypeptide, as well as homologs and analogs thereof. Vascular endothelial growth factor (VEGF) is a 24-42 kDa dimeric glycoprotein that mediates vasodilation, increased vascular permeability, and endothelial cell mitogenesis. The VEGF-Ax polypeptide is a new type of isoform of VEGF-A that is formed as a result of a translational readthrough of VEGF-A mRNA. VEGF-Ax has anti-angiogenic activity, rather than the angiogenic activity provided by VEGF-A. Furthermore, VEGF-Ax isoforms have been shown to have potent anti-angiogenic activity both alone and when added together with VEGF-A, as shown in FIG. 1. A scheme comparing the generation of VEGF-A, VEGF-Ab, and VEGF-Ax from mRNA, and the resulting polypeptides, is shown in FIG. 2.

The VEGF-Ax polypeptide includes a region having at least 85% identity to SEQ ID NO: 1 and having an amino terminus comprising a region having at least 85% identity to SEQ ID NO: 2. The amino acid sequence SAGLEEGASL RVSGTRSLTR KD (SEQ ID NO: 1) corresponds to the Ax peptide, which is found at or near the carboxyl terminal region of the polypeptide, while the amino acid sequence MNFLLSWVHW SLALLLYLHH AKWSQAAPMA EGGQKPHEVV KFMDVYQRSF CRPIETLVDI FQEYPDEIEF IFKPSCVPLM RCGGCCNDES LECVPTEEFN ITMQIMRIKP (SEQ ID NO: 2) corresponds to exons 1-5 of VEGF-A, present at or near the amino terminal region of the polypeptide, which are present in virtually all VEGF isoforms, including VEGF-Ax isoforms. Analysis of the Ax peptide in various mammalian species has shown that this sequence is conserved between various different species. For example, the conservation present in various different species is shown in Table 1.

TABLE 1
Comparison of Coding Sequence Homology for Ax polypeptides

| | | | Downstream 3′ UTR |
|---|---|---|---|
| | Coding | Ax | (66 nt after distal |
| | Sequence | region | stop codon) |
| H. sapiens vs. B. taurus | 95% | 91% | 9% |
| B. taurus vs. M. musculus | 89% | 78% | 9% |
| H. sapiens vs. M. musculus | 90% | 74% | 5% |

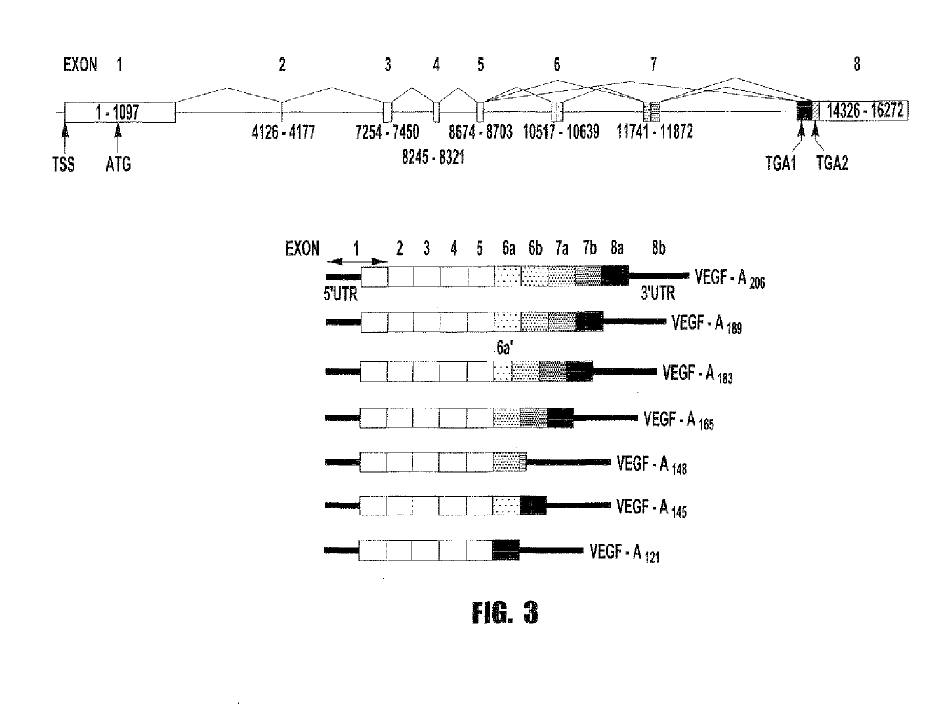

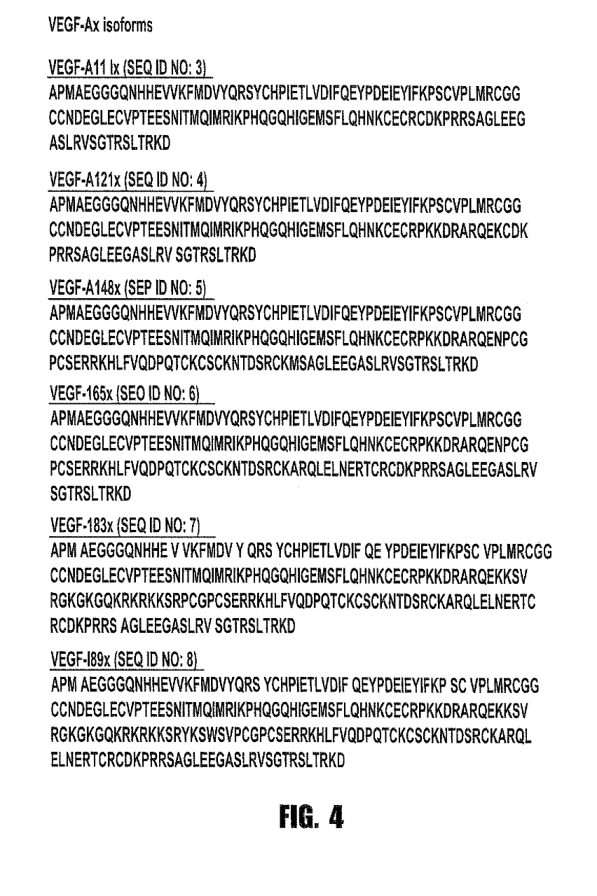

The region between the peptide corresponding to exons 1-5 and the Ax peptide varies substantially from one isoform of VEGF-Ax to another, depending on how exons 6-8, and portions thereof, are spliced together. Various different isoforms of VEGF-A are shown in FIG. 3, which shows isoforms ranging in size from 121 amino acids to 206 amino acids. The amino acid sequence for various different isoforms of VEGF-Ax are provided in FIG. 4. Examples of VEGF-Ax isoforms include VEGF-A111x (SEQ ID NO: 3), VEGF-A121x (SEQ ID NO: 4), VEGF-A148x (SEQ ID NO: 5), VEGF-A165x (SEQ ID NO: 6), VEGF-A183x (SEQ ID NO: 7), and VEGF-A189x (SEQ ID NO: 8). Different embodiments of the invention can make use of any of the VEGF-Ax polypeptides shown in FIG. 4, as well as homologs and analogs thereof.

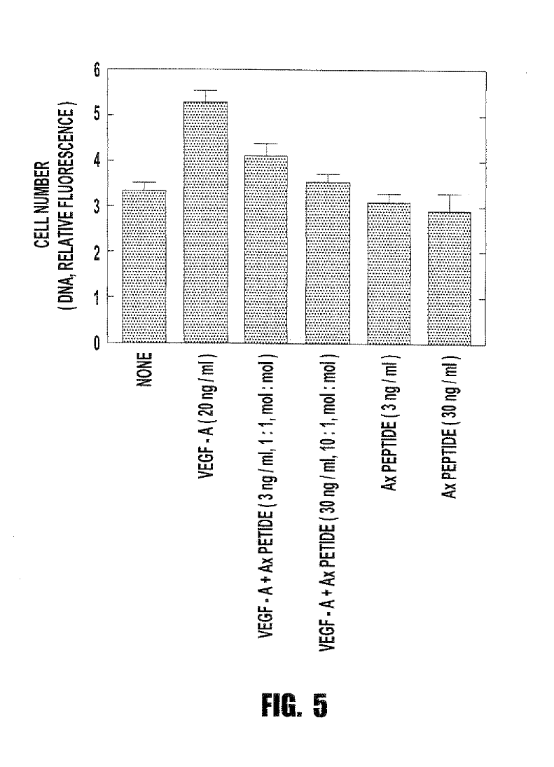

Another aspect of the invention provides an isolated Ax polypeptide, and homologs and analogs thereof. The inventors have surprisingly shown that the isolated Ax polypeptide, lacking the remainder of the VEGF-A polypeptide, also provides substantial anti-angiogenic activity. The anti-angiogenic activity of the Ax polypeptide (SEQ ID NO: 1) is shown in FIG. 5, which shows the anti-angiogenic activity of the Ax polypeptide alone and when used together with angiogenic VEGF-A. In some embodiments, the Ax polypeptide has from 22 to 50 amino acids and includes a region having at least 85% identity to SEQ ID NO: 2, while in other embodiments the polypeptide has from 22 to 30 amino acids and includes a region having at least 85% identity to SEQ ID NO: 2.

In further embodiments, a fragment of the isolated Ax polypeptide (SEQ ID NO: 1) is used. A fragment can be a portion of the Ax polypeptide including from 5 to 20 amino acids, or any intermediate range therein, such as 6-15 or 8-12 amino acids. Individual hexamers of Ax peptide at the beginning, middle, and end of the peptide, particularly the C-terminus six amino acids SLTRKD (SEQ ID NO: 11), may exhibit significant anti-angiogenic activity and present certain advantages such as cost of production and ability to cross the blood-brain barrier for treatment of brain tumors such as glioblastoma.

The angiogenic and anti-angiogenic activity of VEGF-Ax, the Ax polypeptide, and various antibodies described herein can be determined using techniques known in the art. For example, anti-angiogenic activity can be determined by looking at a VEGF-A activity and comparing the inhibition or reduction of such activity when the anti-angiogenic polypeptide is used. Likewise, angiogenic activity can be determined by comparing the activity to VEGF-A activity using the same assay. One can use any isoform of VEGF-A. For example, one can use the human umbilical vein endothelial cell (HUVEC) proliferation assay using VEGF-A165.

The ability of the VEGF-Ax polypeptides, Ax polypeptides, and the antibodies described herein to influence angiogenesis can also be determined using a number of known in vivo and in vitro assays. Such assays are disclosed in Jain et al., Nature Medicine 3, 1203-1208(1997), the disclosure of which is herein incorporated by reference. For example, assays for the ability to inhibit angiogenesis in vivo include the chick chorioallantoic membrane assay and mouse, rat or rabbit corneal pocket assays. See, Polverini et al., Methods Enzymol. 198: 440-450 (1991). In the corneal pocket assays, tumor tissue, cell suspension, or growth factor of choice is implanted into the cornea of the test animal in the form of a corneal pocket. The potential angiogenesis inhibitor is applied to the corneal pocket and the corneal pocket is routinely examined for neovascularization.

By “homologs” what is meant is peptides closely corresponding to the sequences identified for VEGF-Ax and the Ax peptide, including sequences from other mammalian species that are substantially homologous at the overall protein (i.e., mature protein) level to human VEGF-Ax or Ax, so long as such homologous peptides retain their respective known activities. Various levels of homology, from 55% to 99%, are described herein. For example, preferred levels of homology include 75%, 85%, 90%, and 95%.

By “analogs” is meant peptides which differ by one or more amino acid alterations, which alterations, e.g., substitutions, additions or deletions of amino acid residues, do not abolish the properties of the relevant peptides, such as their ability to inhibit angiogenesis. An analog may comprise a peptide having a substantially identical amino acid sequence to a peptide provided herein and in which one or more amino acid residues have been conservatively or non-conservatively substituted. Examples of conservative substitutions include the substitution of a non-polar (hydrophobic) residue such as isoleucine, valine, leucine or methionine for another. Likewise, the present invention contemplates the substitution of one polar (hydrophilic) residue such as between arginine and lysine, between glutamine and asparagine, and between glycine and serine. Additionally, the substitution of a basic residue such as lysine, arginine or histidine for another or the substitution of one acidic residue such as aspartic acid or glutamic acid for another is also contemplated. Examples of non-conservative substitutions include the substitution of a non-polar (hydrophobic) residue such as isoleucine, valine, leucine, alanine, methionine for a polar (hydrophilic) residues such as cysteine, glutamine, glutamic acid, lysine and/or a polar residue for a non-polar residue.

The phrase “conservative substitution” also includes the use of chemically derivatized residues in place of a non-derivatized residues as long as the peptide retains the requisite activity (e.g., anti-angiogenic activity). Analogs also include the presence of additional amino acids or the deletion of one or more amino acids which do not affect VEGF-Ax or Ax peptide mediated biological activity. For example, analogs of the subject peptides can contain an N- or C-terminal cysteine, by which, if desired, the peptide may be covalently attached to a carrier protein, e.g., albumin. Such attachment can decrease clearing of the peptide from the blood and also decrease the rate of proteolysis of the peptides. In addition, for purposes of the present invention, peptides containing D-amino acids in place of L-amino acids are also included in the term “conservative substitution.” The presence of such D-isomers can help minimize proteolytic activity and clearing of the peptide.

Ordinarily, the conservative substitution variants, analogs, and derivatives of the peptides, will have an amino acid sequence identity to the disclosed sequences of at least about 55%, at least about 65%, at least about 75%, at least about 80%, at least about 85%, at least about 90%, at least about 95%, or at least about 96% to 99%. Identity or homology with respect to such sequences is defined herein as the percentage of amino acid residues in the candidate sequence that are identical with the known peptides, after aligning the sequences and introducing gaps, if necessary, to achieve the maximum percent homology, and not considering any conservative substitutions as part of the sequence identity. N-terminal, C-terminal or internal extensions, deletions, or insertions into the peptide sequence shall not be construed as affecting homology.

### Preparation of Peptides

The VEGF-Ax and Ax polypeptides of the present invention, and homologs, analogs and fragments thereof, may be synthesized by a number of known techniques. For example, the peptides may be prepared using the solid-phase synthetic technique initially described by Merrifield in J. Am. Chem. Soc. 85:2149 2154 (1963). In general, the method comprises the sequential addition of one or more amino acid residues to a growing peptide chain. Normally, either the amino or carboxyl group of the first amino acid residue is protected by a suitable, selectively removable protecting group. A different, selectively removable protecting group is utilized for amino acids containing a reactive side group such as lysine. Other peptide synthesis techniques may be found in M. Bodanszky, et al. Peptide Synthesis, John Wiley & Sons, 2d Ed., (1976) and other references readily available to those skilled in the art. A summary of polypeptide synthesis techniques can be found in J. Stuart and J. D. Young, Solid Phase Peptide Synthesis, Pierce Chemical Company, Rockford, Ill., (1984). Peptides may also be synthesized by solution methods as described in The Proteins, Vol. II. 3d Ed., Neurath, H. et al., Eds., p. 105 237, Academic Press, New York, N.Y. (1976). Appropriate protective groups for use in different peptide syntheses are described in the above-mentioned texts as well as in J. F. W. McOmie, Protective Groups in Organic Chemistry, Plenum Press, New York, N.Y. (1973). The peptides of the present invention can also be prepared by chemical or enzymatic cleavage from the entire or larger portions of VEGF polypeptides.

A preferred method of solid phase peptide synthesis entails attaching the protected or derivatized amino acid to an inert solid support through its unprotected carboxyl or amino group. The protecting group of the amino or carboxyl group is then selectively removed and the next amino acid in the sequence having the complementary (amino or carboxyl) group suitably protected is admixed and reacted under conditions suitable for forming the amide linkage with the residue already attached to the solid support. The protecting group of the amino carboxyl group is then removed from this newly added amino acid residue, and the next amino acid (suitably protected) is then added, and so forth. After all the desired amino acids have been linked in the proper sequence, any remaining terminal and side group protecting groups including the solid support are removed sequentially or concurrently to yield the final peptide.

Additionally, the peptides of the present invention may also be prepared by recombinant DNA techniques. Various recombinant cells such as prokaryotic cells, e.g., E. coli, or other eukaryotic cells, such as yeast or insect cells can produce VEGF-Ax or Ax polypeptides. Methods for constructing appropriate vectors, carrying DNA that codes for VEGF-Ax or Ax polypeptides and suitable for transforming (e.g., E. coli, mammalian cells and yeast cells), or infecting insect cells in order to produce a recombinant VEGF-Ax or Ax polypeptides are well known in the art. See, for example, Ausubel et al., eds. “Current Protocols in Molecular Biology” Current Protocols, 1993; and Sambrook et al., eds. “Molecular Cloning: A Laboratory Manual”, 2nd ed., Cold Spring Harbor Press, 1989.

For most of the amino acids used to build proteins, more than one coding nucleotide triplet (codon) can code for a particular amino acid residue. This property of the genetic code is known as redundancy. Therefore, a number of different nucleotide sequences can code for a particular subject decoy peptide. The present invention also contemplates a deoxyribonucleic acid (DNA) molecule or segment that defines a gene coding for, i.e., capable of expressing, a subject peptide or a subject chimeric peptide from which a peptide of the present invention may be enzymatically or chemically cleaved.

DNA molecules that encode peptides of the present invention can be synthesized by chemical techniques, for example, the phosphotriester method of Matteuccie, et al., J. Am. Chem. Soc. 103:3185 (1981). Using a chemical DNA synthesis technique, desired modifications in the peptide sequence can be made by making substitutions for bases which code for the native amino acid sequence. Ribonucleic acid equivalents of the above described DNA molecules may also be used.

For the purposes of expression of VEGF-Ax or Ax polypeptides, the nucleotide sequences encoding VEGF-Ax or Ax polypeptides and the operably linked transcriptional and translational regulatory signals, are inserted into vectors which are capable of integrating the desired gene sequences into the host cell chromosome. In order to be able to select the cells which have stably integrated the introduced DNA into their chromosomes, one or more markers which allow for selection of host cells which contain the expression vector is used. The marker may provide for prototrophy to an auxotropic host, biocide resistance, e.g., antibiotics, or resistance to heavy metals, such as copper, or the like. The selectable marker gene can either be directly linked to the DNA gene sequences to be expressed, or introduced into the same cell by cotransfection. Additional elements may also be needed for optimal synthesis of single chain binding protein mRNA. These elements may include splice signals, as well as transcription promoters, enhancers, and termination signals.

The DNA molecule being introduced into the cells will preferably be incorporated into a plasmid or viral vector capable of autonomous replication in the recipient host. Preferred prokaryotic plasmids are derivatives of pBr322. Preferred eukaryotic vectors include BPV, vaccinia, SV40, 2-micron circle, etc., or their derivatives. Such plasmids and vectors are well known in the art. Once the vector or DNA sequence containing the construct(s) has been prepared for expression, the expression vector may be introduced into an appropriate host cell by any of a variety of suitable means, such as transformation, transfection, lipofection, conjugation, protoplast fusion, electroporation, calcium phosphate precipitation, direct microinjection, etc.

Host cells to be used in this invention may be either prokaryotic or eukaryotic. Preferred prokaryotic hosts include bacteria such as E. coli, Bacillus, Streptomyces, Pseudomonas, Salmonella, Serratia, etc. The most preferred prokaryotic host is E. coli. Bacterial hosts of particular interest include E. coli K12 strain 294 (ATCC 31446). E. coli X1776 (ATCC 31537), E. coli W3110 (F⁻, lambda⁻, phototropic (ATCC 27325). Under such conditions, the protein will not be glycosylated. The prokaryotic host must be compatible with the replicon and control sequences in the expression plasmid.

However, since natural VEGF-Ax are glycosylated proteins, eukaryotic hosts are preferred over prokaryotic hosts. Preferred eukaryotic hosts are mammalian cells, e.g., human, monkey, mouse and Chinese hamster ovary (CHO) cells, because they provide post-translational modifications to protein molecules including correct folding, correct disulfide bond formation, as well as glycosylation at correct sites. Also yeast cells and insect cells can carry out post-translational peptide modifications including high mannose glycosylation.

A number of recombinant DNA strategies exist which utilize strong promoter sequences and high copy number of plasmids, which can be utilized for production of the desired proteins in yeast and in insect cells. Yeast and insect cells recognize leader sequences on cloned mammalian gene products and secrete mature VEGF-Ax or Ax polypeptides. After the introduction of the vector, the host cells are grown in a selective medium, which selects for the growth of vector-containing cells. Expression of the cloned gene sequence(s) results in the production of VEGF-Ax or Ax polypeptides or fragments thereof.

The expressed proteins are then isolated and purified by any conventional procedure involving extraction, precipitation, chromatography, electrophoresis, or the like, or by affinity chromatography, using, e.g., an anti-VEGF-Ax monoclonal antibodies immobilized on a gel matrix contained within a column. Crude preparations containing said recombinant VEGF-Ax are passed through the column whereby VEGF-Ax will be bound to the column by the specific antibody, while the impurities will pass through. After washing, the protein is eluted from the gel under conditions usually employed for this purpose, i.e. at a high or a low pH, e.g. pH 11 or pH 2.

### Antibodies

In other embodiments of the invention, antibodies specific for a portion of the VEGF-Ax or Ax polypeptide can be prepared. Polyclonal antibodies are heterogeneous populations of antibody molecules that can be obtained from the sera of animals immunized with an antigen. To facilitate generation of the antibodies, the substrate peptides can be coupled to a carrier protein such as KLH as described in Ausubel et al. (1989) Current Protocols in Molecular Biology, John Wiley & Sons, New York. The KLH-antagonist peptide is mixed with Freund's adjuvant and injected into guinea pigs, rats, donkeys and the like or preferably into rabbits.

Monoclonal antibodies can be prepared using peptides obtained from a relevant antigen of a VEGF-Ax or Ax polypeptide using standard hybridoma technology (see e.g. Kohler et al., (1975) Nature 256:495; Hammerling et al., (1981) In Monoclonal Antibodies and T Cell Hybridomas, Elsevier, New York). For example, monoclonal antibodies to VEGF-Ax (and homologs, analogs or fragments thereof) can be raised in Balb/C or other similar strains of mice by immunization with purified or partially purified preparations of the VEGF-Ax polypeptide. The spleens of the mice can be removed, and their lymphocytes fused to a mouse myeloma cell line. After screening of hybrids by known techniques, a stable hybrid will be isolated that produces antibodies against a portion of the VEGF-Ax polypeptides. The activity of the antibodies can be demonstrated by their ability to prevent the binding of radiolabelled VEGF-Ax (or vice versa), or their ability to inhibit the biological activity of VEGF-Ax, e.g. on angiogenesis. Once produced, monoclonal antibodies are tested for recognition of a specific antigen of VEGF-Ax by Western blot or immunoprecipitation analysis (using methods described in Ausubel et al., supra). In order to be useful, a peptide fragment of VEGF-Ax must contain sufficient amino acid residues to define the epitope of the VEGF-Ax or Ax polypeptide being detected.

Monoclonal antibodies of the present invention can be humanized to reduce the immunogenicity for use in humans. One approach is to make mouse-human chimeric antibodies having the original variable region of the murine mAb, joined to constant regions of a human immunoglobulin. Chimeric antibodies and methods for their production are known in the art. See, e.g., Sun et al., Proc. Natl. Acad. Sci. USA 84:214 218 (1987); Better et al., Science 240:1041 1043 (1988). Generally, DNA segments encoding the H and L chain antigen-binding regions of the murine mAb can be cloned from the mAb-producing hybridoma cells, which can then be joined to DNA segments encoding C_{H} and C_{L} regions of a human immunoglobulin, respectively, to produce murine-human chimeric immunoglobulin-encoding genes.

To produce monoclonal antibodies, a host mammal is inoculated with a VEGF-Ax or Ax polypeptide and then boosted. Spleens are collected from inoculated mammals a few days after the final boost. Cell suspensions from the spleens are fused with a tumor cell in accordance with the general method described by Kohler and Milstein (Nature, 1975, 256:495-497). In order to be useful, a peptide fragment must contain sufficient amino acid residues to define the epitope of the VEGF-Ax or Ax polypeptide being detected.

If the fragment is too short to be immunogenic, it may be conjugated to a carrier molecule. Some suitable carrier molecules include keyhole limpet hemocyanin and bovine serum albumin. Conjugation may be carried out by methods known in the art. One such method is to combine a cysteine residue of the fragment with a cysteine residue on the carrier molecule. The peptide fragments may be synthesized by methods known in the art. Some suitable methods are described by Stuart and Young in “Solid Phase Peptide Synthesis,” Second Edition, Pierce Chemical Company (1984).

Purification of the antibodies or fragments can be accomplished by a variety of methods known to those of skill including, precipitation by ammonium sulfate or sodium sulfate followed by dialysis against saline, ion exchange chromatography, affinity or immunoaffinity chromatography as well as gel filtration, zone electrophoresis, etc. (Goding in, Monoclonal Antibodies: Principles and Practice, 2d ed., pp. 104-126, Orlando, Fla., Academic Press).

Alternatively, phage display technology can be utilized to select antibody genes with binding activities towards VEGF-Ax or a portion thereof, either from repertoires of PCR amplified v-genes of lymphocytes from humans screened for possessing anti-VEGF-Ax.

In some embodiments, the antibodies can be covalently attached, either directly or via linker, to a compound which serves as a reporter group, either to facilitate treatment or to permit detection of the presence of the VEGF-Ax or Ax polypeptide. A variety of different types of substances can serve as the reporter group, including but not limited to enzymes, dyes, radioactive metal and non-metal isotopes, fluorogenic compounds, fluorescent compounds, etc. Methods for preparation of antibody conjugates of the antibodies (or fragments thereof) of the invention useful for detection and monitoring are described in U.S. Pat. Nos. 4,671,958; 4,741,900 and 4,867,973.

In some embodiments of the invention, preferred binding epitopes may be identified from a known VEGF-Ax gene sequence and its encoded amino acid sequence and used to generate antibodies with high binding affinity. Also, identification of binding epitopes on VEGF-Ax can be used in the design and construction of preferred antibodies. For example, a DNA encoding a preferred epitope on VEGF-Ax may be recombinantly expressed and used to select an antibody which binds selectively to that epitope. The selected antibodies then are exposed to the sample under conditions sufficient to allow specific binding of the antibody to the specific binding epitope on VEGF-Ax and the amount of complex formed then detected. Specific antibody methodologies are well understood and described in the literature. A more detailed description of their preparation can be found, for example, in Practical Immunology, Butt, W. R., ed., Marcel Dekker, New York, 1984.

The present invention provides antibodies and antibody fragments specific for VEGF-Ax antigens. Antibodies are designed for specific binding, as a result of the affinity of complementary determining region of the antibody for the epitope of the target molecule (e.g., VEGF-Ax). For example, an antibody specific for VEGF-Ax can be an antibody or antibody fragment capable of binding to a VEGF-Ax polypeptide with a specific affinity of between 10⁻⁸ M and 10⁻¹¹ M. In some embodiments, an antibody or antibody fragment binds to VEGF-Ax with a specific affinity of greater than 10⁻⁷M, 10⁻⁸M, 10⁻⁹M, 10⁻¹⁰M, or 10⁻¹¹M, between 10⁻⁸M-10⁻¹¹M, 10⁻⁹M-10⁻¹⁰M, and 10⁻¹⁰M-10⁻¹¹M. In a preferred aspect, specific activity is measured using a competitive binding assay as set forth in Ausubel FM, (1994). Current Protocols in Molecular Biology. Chichester: John Wiley and Sons (“Ausubel”), which is incorporated herein by reference.

In one aspect, the present invention provides an antibody specific to the Ax region of a VEGF-Ax polypeptide capable of binding to a VEGF-Ax and not capable of binding to VEGF-A. Such antibodies are useful because they can distinguish VEGF-Ax isoforms from other types of VEGF that may be present in a biological sample, and can be used to specifically target the VEGF-Ax isoforms. In some embodiments, the antibody is specific for a portion of the peptide having at least 85% sequence identity to amino acid sequence SAGLEEGASLRVSGTR (SEQ ID NO: 9). The antibody can be any of the types of antibodies described herein. For example, in some embodiments, the antibody is a monoclonal antibody, while in other embodiments the antibody is a polyclonal antibody.

In another aspect of the present invention, an antibody specific to the C-terminus region of a VEGF-A polypeptide that is capable of binding to the C-terminus region of a VEGF-A polypeptide and not capable of binding to VEGF-Ax is provided. The C-terminus region of VEGF-A includes the amino acid sequence CDKPRR (SEQ ID NO: 10), which is not present in the VEGF-Ab polypeptide, and is present but not exposed at the C-terminus in VEGF-Ax, as shown in FIG. 2. The epitope of an antigen exposed at a C-terminal position is quite different from the epitope of that antigen when it is present in a non-exposed position within the peptide. Antibodies of this type are therefore capable of distinguishing VEGF-A from both VEGF-Ab and VEGF-Ax. Because VEGF-A is the angiogenic form, while VEGF-Ab and VEGF-Ax both exhibit anti-angiogenic effects, this antibody can be used for diagnostic or prognostic uses, or can be used therapeutically to provide an anti-angiogenic effect. The antibody can be any of the types of antibodies described herein. For example, in some embodiments, the antibody is a monoclonal antibody, while in other embodiments the antibody is a polyclonal antibody.

In some embodiments, negative selection is used to increase the specificity of the antibody being obtained. Negative selection involves the step of removing antibodies that are specific for undesired antigens, such as a portion of a peptide outside of the desired antigenic region. For example, antibody can be generated against the C-terminus epitope of pro-angiogenic isoforms of VEGF-A, CDKPRR (SEQ ID NO: 10). The peptide will be conjugated to KLH carrier protein such that CDKPRR (SEQ ID NO: 10) is at the C-terminus. Antibody can then be produced in rabbits by injecting this conjugated peptide. Negative selection of eliminating non-neo-epitope antibodies will be done using CDKPRRS (SEQ ID NO: 12) peptide. In other words, all the antibodies that recognize CDKPRRS (SEQ ID NO: 12) will be removed and the remaining fraction with antibodies specific for CDKPRR (SEQ ID NO: 10) neo-epitope will be retained. Similarly, after preparation of monoclonal antibodies, individual clones will be tested for binding to CDKPRR (SEQ ID NO: 10) but not to CKKPRRS (SEQ ID NO: 13).

### Diagnosis and Immunoassays

The level of the VEGF-Ax or Ax polypeptide can be determined by an immunoassay. Antibodies specifically reactive with VEGF-Ax or a portion thereof such as enzyme conjugates or labeled derivatives, may be used to detect VEGF-Ax in various biological samples, for example they may be used in any known immunoassays which rely on the binding interaction between an antigenic determinant of a protein and the antibodies. Examples of such assays are radioimmunoassays, enzyme immunoassay (e.g., ELISA), immunofluorescence, immunoprecipitation, latex agglutination, hemagglutination, and histochemical tests.

In some embodiments, an ELISA immunoassay is used. The term “ELISA” includes an enzyme-linked immunosorbent assay that employs an antibody or antigen bound to a solid phase and an enzyme-antigen or enzyme-antibody conjugate to detect and quantify the amount of an antigen (e.g., VEGF-Ax) or antibody present in a sample. A description of the ELISA technique is found in Chapter 22 of the 4th Edition of Basic and Clinical Immunology by D. P. Sites et al., 1982, published by Lange Medical Publications of Los Altos, Calif. and in U.S. Pat. Nos. 3,654,090; 3,850,752; and 4,016,043, the disclosures of which are herein incorporated by reference. ELISA is an assay that can be used to quantitate the amount of VEGF-Ax in a sample. In particular, ELISA can be carried out by attaching on a solid support (e.g., polyvinylchloride) an antibody specific for an antigen or protein of interest. Cell extract or other sample of interest such as urine or blood can be added for formation of an antibody-antigen complex, and the extra, unbound sample is washed away. An enzyme-linked antibody, specific for a different site on the antigen is added. The support is washed to remove the unbound enzyme-linked second antibody. The enzyme-linked antibody can include, but is not limited to, alkaline phosphatase. The enzyme on the second antibody can convert an added colorless substrate into a colored product or can convert a non-fluorescent substrate into a fluorescent product. The ELISA-based assay method provided herein can be conducted in a single chamber or on an array of chambers and can be adapted for automated processes.

VEGF-Ax or Ax polypeptide levels can also be determined using an immunoassay that includes the step of contacting the sample with an antibody that is specific for the VEGF-Ax or Ax polypeptide. Antibodies specific for VEGF-Ax or the Ax polypeptide that are used in the methods of the invention may be obtained from scientific or commercial sources. Alternatively, isolated native VEGF-Ax or recombinant VEGF-Ax may be utilized to prepare antibodies, monoclonal or polyclonal antibodies, and immunologically active fragments (e.g., a Fab or (Fab)₂ fragment), an antibody heavy chain, an antibody light chain, humanized antibodies, a genetically engineered single chain Fᵥ molecule (Ladne et al., U.S. Pat. No. 4,946,778), or a chimeric antibody, for example, an antibody which contains the binding specificity of a murine antibody, but in which the remaining portions are of human origin. Antibodies including monoclonal and polyclonal antibodies, fragments and chimeras, may be prepared using methods known to those skilled in the art. Preferably, antibodies used in the methods of the invention are reactive against VEGF-Ax or the Ax polypeptide if they bind with a Kₐ of greater than or equal to 10⁷ M. In some embodiments of the invention, the immunoassay includes the step of contacting the sample with a polyclonal antibody that is specific for the VEGF-Ax or Ax polypeptide.

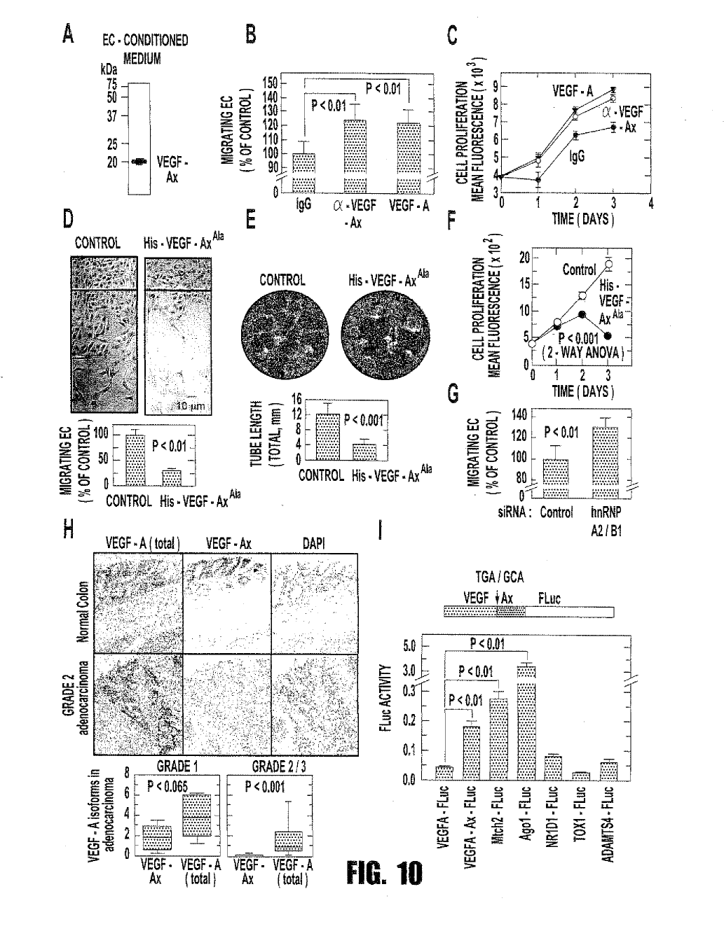

Immunoassays using an antibody specific to the Ax region of a VEGF-Ax polypeptide that is capable of binding to a VEGF-Ax and not capable of binding to VEGF-A can be used for the diagnosis and prognosis of diseases and disorders associated with increased or abnormal angiogenesis. Because VEGF-Ax has anti-angiogenic activity, a decrease in its expression can be a sign that excessive angiogenesis is taking place, as often occurs in cancer. For example, as described herein, VEGF-Ax expression is markedly reduced in cancerous tissues from grade 2 or 3 adenocarcinoma of colon (FIG. 10H). Accordingly, in one aspect, the present invention provides a method of prognosis of a disease or disorder associated with increased angiogenesis in a subject. The first step of the method involves determining the level of VEGF-Ax in a biological sample from a subject by an immunoassay (e.g., an ELISA or immunofluorescence of a tissue sample) using an antibody specific to the Ax region of a VEGF-Ax polypeptide that is capable of binding to a VEGF-Ax and not capable of binding to VEGF-A. Next, the level of VEGF-Ax is compared to a predetermined value based on levels of VEGF-Ax in comparable biological samples obtained from one or more control subjects. Finally, this comparison is used to provide a prognosis of increased severity of the disease or disorder for the subject if the level of VEGF-Ax in the subject is lower than the predetermined value. A variety of diseases associated with increased angiogenesis are described herein. For example, one disease associated with increased angiogenesis is cancer.

“Biological sample” as used herein is meant to include any biological sample from a subject where the sample is suitable for analysis of one or more biomarkers. Suitable biological samples for determining the levels of VEGF-Ax in a subject include but are not limited to bodily fluids such as blood-related samples (e.g., whole blood, serum, plasma, and other blood-derived samples), urine, sputum, cerebral spinal fluid, bronchoalveolar lavage, tears, and the like. In some embodiments, the biological sample is a blood, plasma, or serum sample. Another example of a biological sample is a tissue sample. VEGF-Ax levels can be assessed either quantitatively or qualitatively, usually quantitatively. The levels of VEGF-Ax can be determined either in vitro or ex vivo. A biological sample can be obtained from a subject by any known means including needle stick, needle biopsy, swab, and the like. In an exemplary method, the biological sample is a blood sample, which may be obtained for example by venipuncture.

A biological sample may be fresh or stored. Biological samples may be or have been stored or banked under suitable tissue storage conditions. The biological sample may be a bodily fluid expressly obtained for the assays of this invention or a bodily fluid obtained for another purpose which can be subsampled for the assays of this invention. Preferably, biological samples are either chilled or frozen shortly after collection if they are being stored to prevent deterioration of the sample.

The sample may be pretreated as necessary by dilution in an appropriate buffer solution, heparinized, concentrated if desired, or fractionated by any number of methods including but not limited to ultracentrifugation, fractionation by fast performance liquid chromatography (FPLC), or precipitation with dextran sulfate or other methods. Any of a number of standard aqueous buffer solutions at physiological pH, such as phosphate, Tris, or the like, can be used.

Control values used in diagnostic or prognostic methods are based upon the level of VEGF-Ax in comparable samples obtained from a reference cohort. In certain embodiments, the reference cohort is the general population. For example, the reference cohort can be a select population of human subjects. In certain embodiments, the reference cohort is comprised of individuals who have not previously had any signs or symptoms indicating the presence of a disease or disorder associated with increased or decreased angiogenesis. In certain embodiments, the reference cohort is comprised of individuals, who if examined by a medical professional would be characterized as free of symptoms of disease.

Angiogenesis-Related Disease and Disorders

Angiogenesis can play a role in a variety of diseases and disorders. These diseases and disorders can be divided into two groups: diseases involving increased angiogenesis, and diseases involving decreased angiogenesis. Diseases and disorders involving increased angiogenesis, abnormal angiogenesis, or where decreased angiogenesis would be beneficial, include retinal neovascularization, hemangioma, solid tumors, metastasis, psoriasis, neovascular glaucoma, diabetic retinopathy, macular degeneration, arthritis (e.g., rheumatoid arthritis), endometriosis, retinopathy of prematurity (ROP), gingivitis, and pre-eclampsia. Abnormal angiogenesis is evidenced by the formal of abnormal blood vessels, which are heterogeneous with regard to organization, unevenly distributed, and chaotic. They generally exhibit a serpentine or tortuous course, branch irregularly and form arterio-venous shunts. Abnormal blood vessels may also be thin-walled and leaky

Prevention of solid tumor growth is of particular interest. Examples of cancers that typically involve solid tumor growth include neoplasms of the central nervous system such as, but again not necessarily limited to glioblastomas, astrocytomas, neuroblastomas, meningiomas, ependymomas; cancers of hormone-dependent tissues such as prostate, testicles, uterus, cervix, ovary, mammary carcinomas including but not limited to carcinoma in situ, medullary carcinoma, tubular carcinoma, invasive (infiltrating) carcinomas and mucinous carcinomas; melanomas, including but not limited to cutaneous and ocular melanomas; cancers of the lung which at least include squamous cell carcinoma, spindle carcinoma, small cell carcinoma, adenocarcinoma and large cell carcinoma; and cancers of the gastrointestinal system such as esophageal, stomach, small intestine, colon, colorectal, rectal and anal region which at least include adenocarcinomas of the large bowel.

In other embodiments, the disease of interest involves decreased angiogenesis, or diseases where increased angiogenesis would be beneficial. Such diseases include coronary artery disease, peripheral artery disease, and ischemic stroke, all of which involved systemic atherosclerosis that occludes blood flow. The use of pro-angiogenic therapies to treat such diseases is described by Mac Gabhann, et al., Wiley Interdiscip Rev Syst Biol Med. 2, 694-707 (2010).

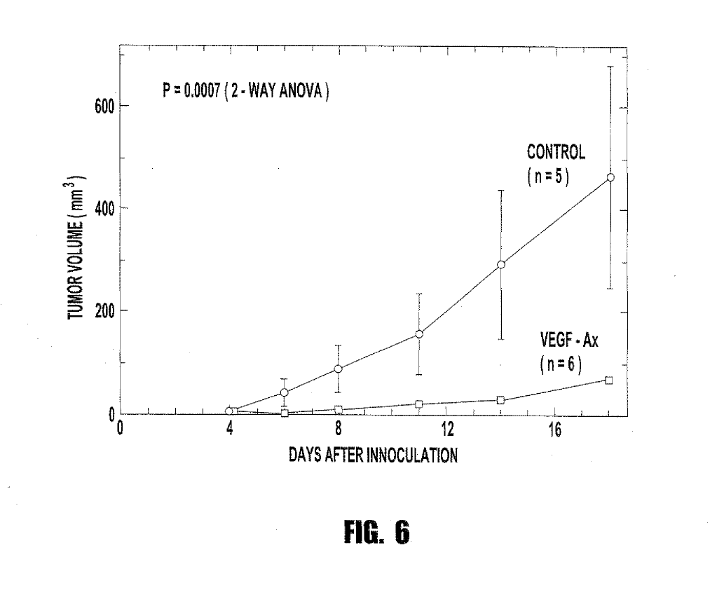

Some aspects of the invention provide methods for treating diseases involving increased angiogenesis. These methods can make use of VEGF-Ax polypeptides, Ax polypeptides, and certain antibodies, which may or may not be administered together with a pharmaceutically acceptable carrier. One of these is a method of decreasing angiogenesis by administering to a subject a therapeutically effective amount of a VEGF-Ax polypeptide, or a homolog or analogs thereof. In some embodiments, the VEGF-Ax polypeptide includes a region having at least 85% identity to SEQ ID NO: 1 and an amino terminus comprising a region having at least 85% identity to SEQ ID NO: 2. The VEGF-Ax polypeptide used can be any of isoform of VEGF-Ax that further includes the Ax polypeptide. For example, in some embodiments, the VEGF-Ax polypeptide has at least 85% identity to SEQ ID NO: 4 (VEGF-A121x), while in other embodiments the VEGF-Ax polypeptide has at least 85% identity to SEQ ID NO: 6 (VEGF-A165x). The subject may have been diagnosed with any disease associated with increased angiogenesis, however, in some embodiments, the subject has been diagnosed as having cancer, and in particular cancer involving a solid tumor requiring additional angiogenesis. The effectiveness of VEGF-Ax in treating cancer is demonstrated in FIG. 6, which shows that injection of VEGF-Ax substantially decreased tumor volume in mice bearing tumor cells.

Another aspect of the invention provides a method of decreasing angiogenesis by administering to a subject a therapeutically effective amount of an Ax polypeptide, or a homolog or analog thereof. In some embodiments, the Ax polypeptide has from 22 to 50 amino acids and includes a region having at least 85% identity to SEQ ID NO: 2, while in other embodiments, the Ax polypeptide has from 22 to 30 amino acids. In a further embodiment, the Ax polypeptide has from 22 to 25 amino acids. As with the VEGF-Ax polypeptide, in some embodiments, the subject has been diagnosed as having cancer, and in particular cancer involving a solid tumor requiring additional angiogenesis.

Yet another method for treating a disease associated with increased angiogenesis involves a method of decreasing angiogenesis in a subject that includes administering a therapeutically effective amount of an antibody specific to the C-terminus region of a VEGF-A polypeptide that is capable of binding to the C-terminus region of a VEGF-A polypeptide and not capable of binding to VEGF-Ax. The method can involve use of any of the types of antibodies described herein, such as monoclonal antibodies, polyclonal antibodies, humanized antibodies, or antibody fragments. In some embodiments, the subject has been diagnosed as having cancer, and in particular cancer involving a solid tumor requiring additional angiogenesis.

Another aspect of the invention provides a method of stimulating angiogenesis in a subject. Stimulating angiogenesis can be useful for treating diseases or disorders involving systemic atherosclerosis that occludes blood flow. The method of stimulating angiogenesis can include administering a therapeutically effective amount of an antibody specific to the Ax region of a VEGF-Ax polypeptide that is capable of binding to VEGF-Ax and not capable of binding to VEGF-A. Because the VEGF-Ax polypeptide has an anti-angiogenic effect, and because angiogenesis reflects a balance between competing factors, use of an antibody to disable all or a portion of the VEGF-Ax polypeptide present has the overall result of stimulating angiogenesis. Examples of diseases that can be treated with an antibody specific for VEGF-Ax include coronary artery disease, peripheral artery disease, and ischemic stroke. The method can involve use of any of the types of antibodies described herein, such as monoclonal antibodies, polyclonal antibodies, humanized antibodies, or antibody fragments.

### Administration of Polypeptides

The present invention also contemplates pharmaceutical formulations for administration to a subject (e.g., a human), which comprise as the active agent the VEGF-Ax polypeptide, the Ax polypeptide, and the antibodies described herein. In such pharmaceutical and medicament formulations, the active agent is preferably utilized together with one or more pharmaceutically acceptable carrier(s) and optionally any other therapeutic ingredients. The carrier(s) must be pharmaceutically acceptable in the sense of being compatible with the other ingredients of the formulation and not unduly deleterious to the recipient thereof. The active agent is provided in an amount effective to achieve the desired pharmacological effect, as described above, and in a quantity appropriate to achieve the desired daily dose.

Typically, the polypeptides (e.g., antibodies) of the present invention will be suspended in a sterile saline solution for therapeutic uses. The pharmaceutical compositions may alternatively be formulated to control release of active ingredient (VEGF-Ax, Ax polypeptide, or antibody) or to prolong its presence in a patient's system. Numerous suitable drug delivery systems are known and include, e.g., implantable drug release systems, hydrogels, hydroxymethylcellulose, microcapsules, liposomes, microemulsions, microspheres, and the like. Controlled release preparations can be prepared through the use of polymers to complex or adsorb the molecule according to the present invention. For example, biocompatible polymers include matrices of poly(ethylene-co-vinyl acetate) and matrices of a polyanhydride copolymer of a stearic acid dimer and sebaric acid. The rate of release of the molecule according to the present invention, i.e., of an antibody or antibody fragment, from such a matrix depends upon the molecular weight of the molecule, the amount of the molecule within the matrix, and the size of dispersed particles.

The pharmaceutical composition of this invention may be administered by any suitable means, such as orally, topically, intranasally, subcutaneously, intramuscularly, intravenously, intra-arterially, intraarticulary, intralesionally or parenterally. Ordinarily, intravenous (i.v.), intraarticular, topical or parenteral administration will be preferred. When administered, the peptide compositions may be combined with other ingredients, such as carriers and/or adjuvants. The peptides may also be covalently attached to a protein carrier, such as albumin, so as to decrease metabolic clearance of the peptides.

It will be apparent to those of ordinary skill in the art that the therapeutically effective amount of the molecule according to the present invention will depend, inter alia upon the administration schedule, the unit dose of molecule administered, whether the molecule is administered in combination with other therapeutic agents, the immune status and health of the patient, the therapeutic activity of the molecule administered and the judgment of the treating physician.

Although an appropriate dosage of a molecule of the invention varies depending on the administration route, age, body weight, sex, or conditions of the subject, and should be determined by the physician in the end, in the case of oral administration, the daily dosage can generally be between about 0.01 mg to about 500 mg, preferably about 0.01 mg to about 50 mg, more preferably about 0.1 mg to about 10 mg, per kg body weight. In the case of parenteral administration, the daily dosage can generally be between about 0.001 mg to about 100 mg, preferably about 0.001 mg to about 10 mg, more preferably about 0.01 mg to about 1 mg, per kg body weight. The daily dosage can be administered, for example in regimens typical of 1-4 individual administration daily. Other preferred methods of administration include intraarticular administration of about 0.01 mg to about 100 mg per kg body weight. Various considerations in arriving at an effective amount are described, e.g., in Goodman and Gilman's: The Pharmacological Bases of Therapeutics, 8th ed., Pergamon Press, 1990; and Remington's Pharmaceutical Sciences, 17th ed., Mack Publishing Co., Easton, Pa., 1990.

The peptides of the present invention as active ingredients are dissolved, dispersed or admixed in an excipient that is pharmaceutically acceptable and compatible with the active ingredient as is well known. Suitable excipients are, for example, water, saline, phosphate buffered saline (PBS), dextrose, glycerol, ethanol, or the like and combinations thereof. Other suitable carriers are well known to those skilled in the art. In addition, if desired, the composition can contain minor amounts of auxiliary substances such as wetting or emulsifying agents, pH buffering agents.

The following example is included for purposes of illustration and is not intended to limit the scope of the invention.

### EXAMPLE

### Example 1

### Programmed Translational Readthrough Generates Anti-angiogenic VEGF-Ax

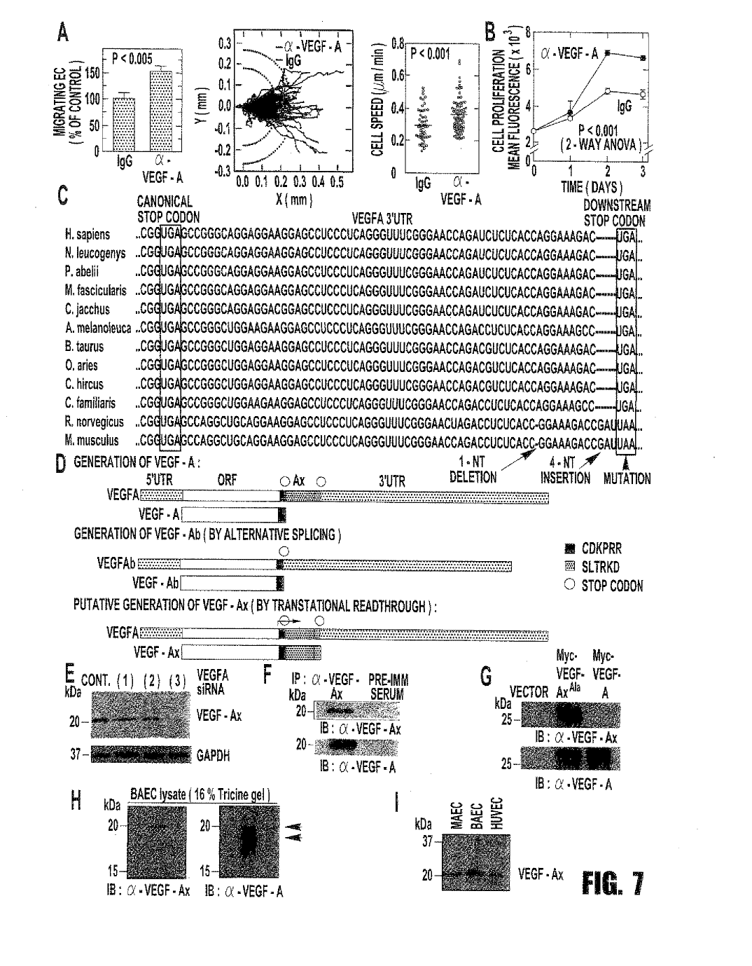

The possible paracrine activity of endothelial cell (EC)-derived VEGF-A on EC migration and proliferation, was investigated. The paracrine activity of EC-derived VEGF-A on EC migration is essential for endothelial wound-healing response and angiogenesis. D. H. Ausprunk, J. Folkman, Microvasc. Res. 14, 53-65 (1977); Schwartz et al., Lab. Invest. 38, 568-580 (1978). Unexpectedly, VEGF-A-neutralizing antibody enhanced migration of bovine aortic ECs as indicated by the number of migrating cells in a razor-wound assay, as well as by mean speed and root-mean-square displacement (FIG. 7A). Likewise, the antibody increased EC proliferation determined fluorimetrically as total cell DNA (FIG. 7B). These results indicate that ECs secrete an anti-angiogenic isoform of VEGF-A.

To test for the possible expression of VEGF-Ab the inventors sequenced 74 VEGFA cDNA clones from bovine aortic ECs (and 6 from mouse aortic ECs), but did not find VEGF-Ab-specific mRNA or any alternatively-spliced VEGFA mRNA that can generate anti-angiogenic isoforms. Instead, only mRNAs corresponding to pro-angiogenic VEGF-A164 and -Al20 were identified. Reverse transcriptase PCR, using primers specifically designed to differentiate VEGF-Ab from pro-angiogenic VEGF-A isoforms, also did not detect VEGF-Ab-specific mRNA in ECs.

The paradoxical presence of anti-angiogenic VEGF-A in the absence of VEGF-Ab-specific mRNA suggested the possibility of a novel anti-angiogenic VEGF-A isoform generated by an unknown mechanism from mRNA encoding the pro-angiogenic isoform. A critical clue was offered by the sequence of the VEGFA mRNA 3′ untranslated region (UTR). In the human VEGFA 3′UTR an evolutionarily conserved UGA stop codon was observed 63 nt downstream and in-frame to the canonical stop codon (FIG. 7C). Importantly, a 4-nt insertion in rodent 3′UTRs is offset by a 1-nt deletion thereby preserving the in-frame nature of the stop codons. Moreover, a mutation in the downstream rodent stop codon generates the alternate UAA stop codon, providing additional evolutionary support for the significance of the in-frame stop codon. The amino acid sequence potentially encoded by the region between the stop codons, but not the sequence beyond the downstream stop codon, is likewise conserved in mammals. Based on these observations it was hypothesized that VEGFA mRNA translation might extend beyond its canonical stop codon, terminating at the downstream, in-frame stop codon, generating a 21-(22-in rodents) amino acid extension. This translational readthrough event in primate or bovine cells would generate a VEGF-A isoform with S/RLTRKD (SEQ ID NO: 14) at the C-terminus, potentially conferring anti-angiogenic activity to a protein generated from “pro-angiogenic” VEGFA mRNA (FIG. 7D).

To investigate this hypothesis, an antibody was generated against the unique peptide, SAGLEEGASLRVSGTR (SEQ ID NO: 9), generated by the proposed translational readthrough event, and not present in VEGF-Ab or any other protein (or in-silico translated mRNA). A 20-kDa protein corresponding to the predicted size of the readthrough product, designated VEGF-Ax (for extended), was identified in lysates from bovine ECs; expression was inhibited by transfection with three different VEGFA-specific siRNAs, consistent with a VEGF-A isoform (FIG. 7E). Protein immunoprecipitated with VEGF-Ax antibody was recognized by an anti-VEGF-A antibody that targets the N-terminus, establishing VEGF-Ax as a novel VEGF-A isoform (FIG. 7F). To validate antibody specificity, HEK293 cells were transfected with Myc-tagged VEGFA cDNA up to the downstream stop codon, and in which the canonical stop codon is mutated to GCA (encoding Ala) to ensure efficient generation of the modified, extended isoform, denoted VEGF-Ax^{Ala}. Anti-VEGF-Ax antibody recognized VEGF-Ax^{Ala} but not VEGF-A generated by transfection of VEGFA cDNA (FIG. 7G). Also, anti-VEGF-Ax antibody did not recognize recombinant VEGF-Ab. An immunoblot done using 16% Tricine gel and probed with anti-VEGF-A antibody distinguished VEGF-Ax from VEGF-A, the former constituting˜10% of the total; both samples were deglycosylated to minimize confounding effects (FIG. 7H). VEGF-Ax expression also was observed in murine aortic and human umbilical vein ECs (FIG. 71), and in multiple other cell types including macrophages, hepatocytes, keratinocytes, and tumor-derived cells.

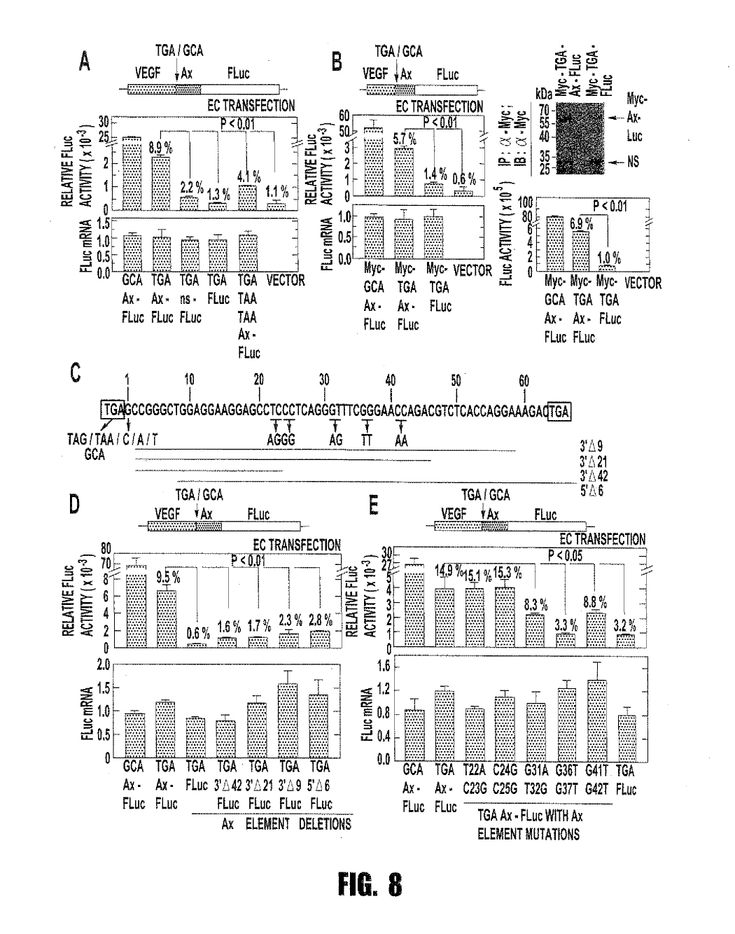

To validate translation readthrough and elucidate the molecular mechanism, a reporter assay was established in which full-length bovine VEGFA coding sequence (corresponding to VEGF-A₁₆₄) up to and including the 63-nt, Ax-specific sequence (termed Ax element) was cloned upstream of and in-frame with firefly luciferase (FLuc). The downstream stop codon was excluded so that readthrough at the canonical stop codon generated a FLuc-containing chimeric protein. ECs transfected with the reporter exhibited substantial FLuc activity, consistent with translation readthrough (FIG. 8A, top). To estimate readthrough efficiency, the upstream TGA stop codon was mutated to GCA (encoding Ala) to maximize FLuc expression; stop codon readthrough efficiency was about 9%. FLuc activity was decreased to near background level when the Ax element was removed or replaced with a non-specific sequence. Enhancement of translational termination efficiency by an in-frame insertion of two stop codons (TAA) immediately downstream of the canonical stop codon substantially reduced FLuc activity, consistent with translational readthrough. Quantitative RT-PCR analysis showed that all constructs were transcribed at similar levels, indicating that differential FLuc activity was due to differences in translation (FIG. 8A, bottom). Readthrough was further validated by mass spectrometric analysis of the product generated by overexpressing VEGFA cDNA containing the Ax element and the canonical stop codon in HEK293 cells. In addition to detection of VEGF-A-specific peptides, selected reaction monitoring revealed a spectrum consistent with RSAGLEEGASLR (SEQ ID NO: 15) peptide (S, serine in place of stop codon) that is specific for the readthrough region; the profile was confirmed by the spectrum obtained from synthetic RSAGLEEGASLR (SEQ ID NO: 15) peptide. Spectra consistent with serine in place of the stop codon were observed in 3 out of 3 independent experiments. Targeted search for potential readthrough peptides containing the other 19 amino acids in place of the stop codon was negative supporting serine insertion during UGA stop codon suppression. Together, these results verify translational readthrough in VEGFA mRNA, and further show that the Ax element is essential for the process, consistent with a programmed translation readthrough (PTR) unprecedented in vertebrate mRNAs.

To investigate if the Ax element drives readthrough in a heterologous context, a construct was engineered containing Myc terminated with a TGA stop codon, upstream and in-frame to the Ax element and FLuc (Myc-Ax-FLuc). Transfection into ECs induced substantial FLuc activity compared to the construct lacking the Ax element or empty vector (FIG. 8B, left). The readthrough product was also detected by immunoblot (FIG. 8B, top-right) and mass spectrometry following immunoprecipitation using anti-Myc-tag antibody. Robust Ax-driven expression was observed when Myc-Ax-FLuc was subjected to in vitro translation in wheat germ extract, formally eliminating a possible requirement for splicing (FIG. 8B, bottom-right). Surprisingly, Ax element-mediated VEGFA readthrough was observed across all three stop codons in transfected ECs, and also following in vitro translation. This result eliminates single stop codon-dependent mechanisms, such as UGA-dependent selenocysteine insertion (S. C. Low, M. J. Berry, Trends Biochem. Sci. 21, 203-208 (1996)), for VEGFA translational readthrough. Similarly, mutation of the first nucleotide after the canonical stop codon (G1) did not affect the readthrough efficiency ruling out context-dependent readthrough mechanisms observed in other systems (Tate et al., Biochemistry(Mosc) 64, 1342-1353 (1999)). Together, these experiments reveal the Ax element as a necessary and sufficient translational readthrough signal in VEGFA mRNA.

To determine the Ax element region necessary for function, deletions from both termini were generated. Deletion of 9, 21, or 42 nt from the 3′ end or 6 nt from the 5′ end substantially reduced FLuc activity suggesting that both termini are required, and possibly the entire element, for readthrough (FIG. 8C, D). To investigate the role of the central domain the inventors generated five pairs of mutations in sequences selected for their conservation in multiple mammalian species. Mutation of nt pairs at positions 31 and 41 partially inhibited readthrough, whereas mutation of the pair at position 36 reduced readthrough to background level (FIG. 8E). Mutation of nt pairs at 22 and 24 were completely ineffective. Thus, essentially the entire length of the Ax element is required to execute readthrough, possibly indicating a relatively large structural element or an interaction with multiple trans-acting factors.

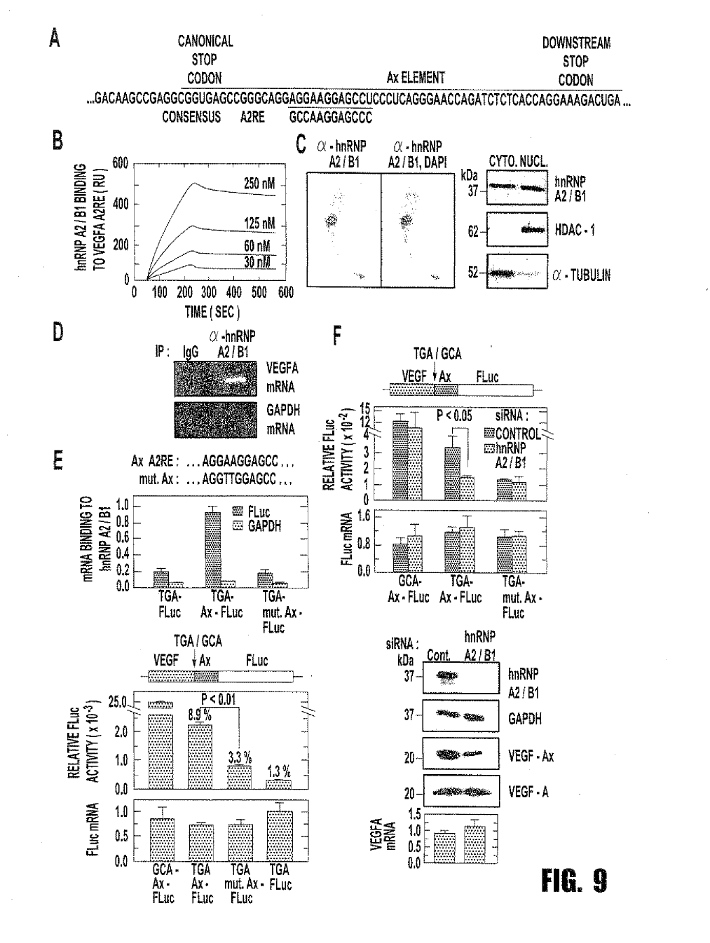

A near-consensus heterogeneous nuclear ribonucleoprotein (hnRNP) A2/B1 recognition element (A2RE) was identified within the Ax element (FIG. 9A). hnRNP A2/B1 is an RNA-binding protein involved in pre-mRNA processing and mRNA transport, and binds the sequence 5′-GCCAAGGAGCC-3′ (SEQ ID NO: 16). Y. He, R. Smith, Cell. Mol. Life Sci. 66, 1239-1256 (2009); Shan et al., J. Neurosci. 23, 8859-8866 (2003). Surface plasmon resonance spectroscopy revealed a high-affinity interaction between hnRNP A2/B1 and a VEGFA RNA segment containing the A2RE, with a dissociation constant (K_{D}) of 19.2±5.6 nM (FIG. 9B). hnRNP A2/B1 is localized primarily in the nucleus; however, cytoplasmic localization was detected in ECs as well (FIG. 9C). An RNA-binding protein immunoprecipitation (RIP) experiment confirmed the intracellular interaction between endogenous hnRNP A2/B1 and VEGFA mRNA (FIG. 9D). To interrogate the interaction specificity, ECs were transfected with a FLuc reporter bearing wild-type, mutant (AA-to-TT), or no Ax element. RIP followed by quantitative RT-PCR analysis showed that hnRNP A2/B1 interacted with wild-type Ax element, but not with the mutant element (FIG. 9E, top). Munro et al., J. Biol. Chem. 274, 34389-34395 (1999). Mutation of the A2RE substantially reduced readthrough without altering mRNA expression (FIG. 8E, bottom 2 panels). The marginal readthough in the absence of hnRNP A2/B1 binding suggests that other factors might contribute, consistent with the requirement for the entire Ax element, not just the A2RE. siRNA-mediated knockdown of hnRNP A2/B1 in ECs reduced readthrough of a VEGFA-Ax-FLuc reporter without inhibiting FLuc activity of a construct with GCA in place of the canonical stop codon or with mutant Ax element (FIG. 9F). The treatment did not affect FLuc mRNA level as shown by qRT-PCR. Also, hnRNP A2/B1 knockdown reduced endogenous expression of VEGF-Ax without affecting total VEGF-A protein or mRNA in ECs (FIG. 9F). These observations show the important regulatory role of hnRNP A2/B1 as a trans-acting factor promoting PTR of VEGFA mRNA.

Immunoblot analysis of EC-conditioned medium showed VEGF-Ax secretion, suggesting a potential paracrine function (FIG. 10A). Inhibition of extracellular VEGF-Ax by anti-VEGF-Ax antibody or VEGF-Ax generation by antisense morpholino did not reduce cell viability as determined by propidium iodide and annexin V staining. However, anti-VEGF-Ax antibody increased EC migration and proliferation to an extent comparable to induction by recombinant VEGF-A, consistent with a paracrine, anti-angiogenic activity of endogenous VEGF-Ax (FIG. 10B,C). Planar EC migration, tube formation in Matrigel, and proliferation were markedly inhibited by purified, recombinant His-VEGF-Ax^{Ala} (FIG. 10D-F). Similarly, siRNA-mediated knockdown of hnRNP A2/B1, which decreases VEGF-Ax production, stimulated EC migration (FIG. 10G). These results indicate that VEGF-Ax is the functionally dominant isoform of VEGF-A released by ECs, exerting a potent paracrine, anti-angiogenic activity.

To investigate the physiological significance of VEGF-Ax, its expression in vivo was investigated. VEGF-Ax was detected in tissues from human brain, colon, small intestine, spleen, pancreas, and serum from healthy human subjects. Remarkably, VEGF-Ax expression is markedly reduced in cancerous tissues from grade 2 or 3 adenocarcinoma of colon, despite abundant VEGF-A (FIG. 10H). These results show that VEGF-A translational readthrough occurs in vivo and suggest a possible negative role of VEGF-Ax in tumor progression.

Although there are no reports of PTR, two readthrough events have been described in vertebrate mRNAs, namely, β-globin in rabbits and myelin protein zero in rats. Chittum et al., Biochemistry 37, 10866-10870 (1998); Yamaguchi et al., J. Biol. Chem. 287, 17765-17776 (2012). Functional significance of the readthrough product has not been shown in either case. Moreover, cis-acting RNA signals or other regulatory mechanisms have not been elucidated. In the case of β-globin, serine is one of several amino acids recoding the UGA stop codon. Interestingly, a suppressor serine tRNA that recognizes UGA is expressed in mammals. Hatfield et al., Proc. Natl. Acad. Sci. U.S.A. 79, 6215-6219 (1982).

Basal readthrough generated by fidelity errors is estimated to range between 0.01 and 0.1% in mammals. Floquet et al., PLoS Genet. 8, e1002608 (2012). In studies carried out by the inventors, the readthrough efficiency of VEGFA mRNA ranges from 7 to 25%. In viruses, where readthrough events are well-established, efficiencies of 6% in murine leukemia virus and 2-5% in Sindbis have been reported. Houck-Loomis et al., Nature 480, 561-564 (2011). In these cases the nucleotide sequence downstream of the canonical stop codon serves as a signal directing readthrough, establishing them as authentic programmed events distinguishable from translation errors. Firth et al., Nucleic Acids Res. 39, 6679-6691 (2011). The inventors show that the 63-nt Ax element is the cis-acting signal for translation readthrough establishing it as an authentic “programmed” event. Coincidentally, a 63-nt sequence element induces translational readthrough in gag-pol. In this case, a pH-dependent alteration in conformation of a pseudoknot in the sequence element determines readthrough. Wills et al., EMBO J. 13, 4137-4144 (1994). Regulation of VEGFA PTR by hnRNP A2/B1 is particularly noteworthy in that control of translational readthrough by a trans-acting protein has not been reported. The precise molecular mechanism by which the Ax element and hnRNP A2/B1 direct readthrough is not known. It may be that they reduce the interaction of eukaryotic release factor 1 (eRF1) with the stop codon, thereby allowing cognate suppressor tRNAs, or even near-cognate tRNAs, to suppress termination. Hatfield et al., Crit. Rev. Biochem. Mol. Biol. 25, 71-96 (1990) Ramakrishnan and coworkers have observed that “suppression of stop codons by tRNAs requires only that they are able to outcompete release factors; they do not have to be as efficient as cognate tRNAs on sense codons”. Fernandez et al., Nature 500, 107-110 (2013) Interestingly, tRNA^{Ser(Sec)}, which facilitates recoding of UGA by selenocysteine, has been suggested to act as a nonsense suppressor and insert Ser. Chittum et al., Biochemistry 37, 10866-10870 (1998) Moreover, reports of nonsense suppressor tRNAs for all three stop codons in mammals, and the ability of eRF1 to recognize all three stop codons, are consistent with the non-selective nature of Ax-mediated readthrough. Frolova et al., Nature 372, 701-703 (1994).

The discovery of anti-angiogenic VEGF-A isoforms compels a need for cognizance and caution in applying VEGF-A-targeted therapies for treatment of VEGF-A-dependent malignancies, as coincident diminution of VEGF-Ax might exacerbate angiogenesis and tumorigenesis. Moreover, usefulness of VEGF-A as a biomarker might also be re-considered in the light of potential presence of both pro- and anti-angiogenic isoforms. The expression of VEGF-Ax may be an innate mechanism that evolved to limit angiogenesis during tissue homeostasis and reduce pathological angiogenesis. However, in some conditions exemplified by colon adenocarcinoma, a loss of expression of VEGF-Ax might induce angiogenesis and exacerbate pathogenesis. Thus, abnormally low levels of VEGF-Ax in plasma or tissues can serve as a biomarker for prognosis or susceptibility to treatment. Moreover, because of its robust anti-angiogenic activity, VEGF-Ax provides a potential therapeutic agent against diseases characterized by excessive angiogenesis.

### Materials and Methods:

Cell culture. Bovine aortic endothelial cells (ECs) were cultured in DME/Ham's F-12 medium containing 5% fetal bovine serum (FBS) and used in all experiments unless specified otherwise. Human umbilical vein ECs were cultured in EC growth medium with SingleQuots supplements (Lonza). Mouse aortic ECs were cultured in DME/Ham's F-12 medium supplemented with 25 μg/ml EC growth supplement (Sigma), 10 U/ml heparin, and 10% FBS. HEK293 cells were cultured in DMEM medium containing 10% FBS. Cells were maintained at 37° C. in a humidified atmosphere with 5% CO₂.

Antibodies. Polyclonal anti-VEGF-Ax antibody was generated by injecting into rabbits synthetic KLH-conjugated peptide, SAGLEEGASLRVSGTR (SEQ ID NO: 9); the same peptide was used for affinity purification. Anti-hnRNP A2/B1 was from Abcam (for immunofluorescence microscopy) or from Santa Cruz (clone EF-67, for immunoprecipitation and immunoblot analysis). Anti-VEGF-A (N-terminus), neutralizing anti-VEGF-A (clone JH121), and horseradish peroxidase-conjugated secondary antibodies were from Thermo Fisher; anti-VEGF-Ab was from R&D; anti-CD31 was from Dako; anti-Myc-tag was from Cell Signaling; anti-GAPDH and anti-α-tubulin were from Sigma®; anti-HDAC-1 was from BioVision™; IgG from Santa Cruz, and Alexa Fluor-conjugated secondary antibody was from Molecular Probes®.

Cell migration. Cell migration was measured by razor-wound method. Confluent bovine aortic EC cultures maintained in serum-free medium for 24 h were wounded by gently pressing a razor through the cell layer to mark the wound line and then drawn through the monolayer to remove cells on one side of the line. Migration was allowed for 24 h in serum-free medium containing 1 mg/ml of bovine serum albumin and IgG or anti-VEGF-A or—VEGF-Ax antibodies (5 mg/ml) or His-VEGF-Ax^{Ala} (50 ng/ml). Following fixation and staining with Giemsa-Wright (Sigma®), cells crossing the wound line at two randomly chosen regions 1.5 mm in length were counted by a semi-automated, computer-assisted procedure. To calculate root-mean-square displacement and speed, cells were imaged every 5 min for a 1000 min by a phase-contrast microscope (Leica Microsystems™) equipped with temperature-controlled humidified chamber and motorized x-y stage. Migrating cells were subjected to automatic tracking using “track objects” function in Metamorph and x- and y-coordinates were acquired. All cell migration experiments were analyzed by a person blinded to the treatments.

Cell proliferation. Bovine aortic ECs were allowed to attach for 4 h in 96-well plates (10,000 cells/well) and medium replaced with Opti-MEM (Invitrogen™) containing IgG, or anti-VEGF-A or -VEGF-Ax antibodies (5 μg/ml), recombinant VEGF-A (R&D Systems, 20 ng/ml), or His-VEGF-Ax^{Ala} (50 ng/ml). Total cell DNA was determined fluorometrically using CyQUANT NF Cell Proliferation Assay Kit (Invitrogen™) with excitation at 485 nm and emission at 538 nm (Spectramax Gemini EM).

Apoptosis/cell death assay. Bovine aortic ECs were allowed to attach for 12 h in 6-well plates (500,000 cells/well) and medium replaced with Opti-MEM (Invitrogen™) containing IgG, VEGF-Ax antibody (5 μg/ml), or control or Ax anti-sense morpholino (5′ ACGTCTGGTTCCCGAAACCCTGAGG 3′ (SEQ ID NO: 17), Gene Tools, LLC). Etoposide (10 μM) was used as positive control for apoptosis. After 48 h, cells were harvested, stained with fluorescein isothiocyanate (FITC)-conjugated annexin V and propidium iodide (Invitrogen™), and subjected to flow cytometry (BD FACScan). Results were analyzed using WinMDI 2.8 software.

Mass spectrometry. Bovine VEGFA₁₆₄ cDNA and the Ax element were cloned in a construct containing an in-frame polyHis-tag. The downstream stop codon was excluded so that readthrough at the canonical stop codon generated a polyHis-tagged chimeric protein. Serum-free conditioned medium was obtained from stably transfected HEK293 cells. His-tagged readthrough product was purified using Ni-NTA agarose (Qiagen™) and subjected to SDS-PAGE electrophoresis. An ˜28 kDa band was cut from the gel stained with Imperial protein stain (Thermo), digested with trypsin, and analyzed by capillary column LC-MS/MS (LTQ-Orbitrap Elite system coupled to a Dionex Ultimate 3000 HPLC fitted with a 15 cm×75 μm i.d. Acclaim Pepmap C18 reverse phase capillary column). Peptides were eluted using 0.5% formic acid and 85% acetonitrile as the mobile phases at a flow rate of 0.3 μl/min. The digest was analyzed in survey and targeted modes. Survey experiments were done using the data-dependent multitask capability of the instrument acquiring full scan mass spectra to interrogate peptide molecular weights and product ion spectra to determine amino acid sequence in successive instrument scans. The data were analyzed by Mascot using all collected CID spectra to search the bovine reference sequence database. The targeted selective reaction monitoring (SRM) experiments involved fragmentation of specific readthrough peptides over the entire course of the LC experiment.

Immunoblot analysis. Cell lysates or conditioned media were denatured and resolved on 4-20% gradient SDS-PAGE. To differentiate canonical VEGF-A and VEGF-Ax on same gel, lysates were subjected to protein deglycosylation mix (New England Biolabs®) and resolved on 16% Tricine gel (Invitrogen™). After transfer, the blots were probed with specific primary antibody followed by HRP-conjugated secondary antibody, and developed using ECL or ECL plus reagent (Amersham™). Nuclear and cytoplasmic fractions were derived from ECs using NE-PER reagent (Thermo Scientific™)

siRNAs. VEGF-A-specific siRNAs target the following sequences in bovine VEGFA mRNA:

(SEQ ID NO: 18)
siRNA 1, 5′ GCTTCCTACAGCATAACAAATGTGA 3′
(SEQ ID NO: 19)
siRNA 2, 5′ GGAGTACCCAGATGAGATT 3′
(SEQ ID NO: 20)
siRNA 3, 5′ ATGTGAATGCAGACCAAAG 3′.

hnRNP A2/B1-specific siRNA targets the following sequence in bovine HNRNPA2/B1

(SEQ ID NO: 21)
mRNA: 5′ GGCTTTGTCTAGACAAGAAATGCAG 3′.

All siRNAs were transfected using Lipofectamine 2000 and expression of target genes determined after 3 days by immunoblot analysis.

### Immunoprecipitation and RNA-Binding Protein Immunoprecipitation (RIP).

Cell extracts were pre-cleared with protein A-Sepharose beads and IgG for 1 h at 4° C. Antibody was added and samples tumbled overnight at 4° C. Immune complexes bound to protein A-Sepharose beads were isolated by centrifugation followed by extensive washing. For immunoprecipitation, protein was extracted using Laemmli buffer. For RIP analysis, RNA bound to immune complexes was isolated by RNeasy Mini Kit (Qiagen™)

VEGFA sequencing. Total RNA was isolated from bovine ECs using RNeasy Mini Kit (Qiagen™). VEGFA cDNA was generated by reverse transcription followed by PCR using SuperScript III One-Step RT-PCR System (Invitrogen™). Primers used were:

(SEQ ID NO: 22)
Forward, 5′ ATGCAAGCTTATGAACTTTCTGCTCTCTTGGG 3′
(SEQ ID NO: 23)
Reverse, 5′ ATGCGGATCCGTCTTTCCTGGTGAGACGTCT 3′

cDNAs were cloned in pGEM-T vector and subjected to capillary sequencing using T7

(SEQ ID NO: 24)
Universal-R1 primer: 5′ TAATACGACTCACTATAGG 3′.

RT-PCR analysis. Total bovine EC RNA or RNA isolated from RIP samples were subjected to reverse transcription and real-time PCR using AgPath-ID One-Step RT-PCR reagent (Ambion). Firefly luciferase (FLuc)-, bovine VEGFA- and GAPDH-specific TaqMan probes (Applied Biosystems) were used. FLuc and VEGFA mRNA levels were normalized by GAPDH mRNA. In RIP experiments, hnRNP A2/B1-bound FLuc mRNA was quantified relative to the FLuc mRNA in input samples. Following VEGFA-specific primers were used for PCR to differentiate between anti-angiogenic VEGF-Ab isoforms and pro-angiogenic VEGF-A isoforms:

(SEQ ID NO: 25)
Forward, 5′ ATGCGGATCAAACCTCACC 3′;
(SEQ ID NO: 26)
Reverse, 5′ GTCTTTCCTGGTGAGACG 3′.

Plasmid construction. pcDNA 3.1 (Invitrogen™) was the backbone vector for all constructs. Bovine VEGFA₁₆₄ cDNA from ECs was cloned with the Ax element in HindIII and BamHI sites. The canonical stop codon separating VEGF-A₁₆₄ coding sequence from Ax element was retained, but the downstream in-frame stop codon was omitted. Firefly luciferase (FLuc) was cloned without its start codon (ATG) between BamHI and NotI. The following linker sequence was added at the 5′ end of FLuc: 5′ GGCGGCTCCGGCG-GCTCCCTCGTGCTCGAG 3′ (SEQ ID NO: 27). In a separate construct, Myc replaced VEGF-A₁₆₄. In all constructs, FLuc was in-frame with VEGFA₁₆₄ or Myc. Mutations were done using GeneArt Site-Directed Mutagenesis System (Invitrogen™). To test candidate genes for translational readthrough, ˜700 nt at the 3′ end of human coding sequences (TOX, 684 nt; ADAMTS4, 741 nt; AGO1, 696 nt; NR1D1, 669 nt; MTCH2, 732 nt) were cloned with inter-stop codon regions upstream to and in-frame with FLuc. Authenticity of all constructs was confirmed by sequencing.

Luciferase-based translational readthrough assay. Plasmids containing chimera of FLuc downstream of test sequences (VEGFA, Myc, TOX, ADAMTS4, AGO1, NR1D1, or MTCH2) were transfected (500 ng/well) into bovine ECs in 24-well plates using Lipo-fectamine 2000. Plasmid expressing Renilla luciferase (RLuc) (50 ng/well) was co-transfected as efficiency control. After 48 h, cells were lysed and FLuc and RLuc activity were measured using Dual-Luciferase Reporter Assay System (Promega™) in Victor³ 1420 Multilabel Plate Counter (Perkin-Elmer). The same constructs were subjected to in vitro coupled transcription-translation (500 ng/reaction) using T_{N}T T7 Coupled Wheat Germ Extract System (Promega™). FLuc activity was measured by Luciferase Assay System (Promega™)

Surface plasmon resonance. Biotinylated RNA (5′ Bio-UGUGACAAGCCGAGGCGGUGAGCCGGGCAGGAGGAAGGAGCCUC 3′ (SEQ ID NO: 28)) containing the putative A2RE of VEGFA mRNA (Dharmacon™) was immobilized on a streptavidin sensor chip in a BIAcore 3000 (GE Health™). Recombinant human hnRNP A2/B1 (Abnova™) was injected at the rate of 20 μl/min for 3 min in a running buffer of 10 mM HEPES (pH 7.4), 150 mM NaCl and 0.005% Surfactant P20, and cells were regenerated using 50 mM NaOH. K_{D} were calculated using Biaevaluation software.

Expression and purification of His-VEGF-Ax^{Ala}. HEK293-6E cells were cultured in serum-free Freestyle 293 expression medium (Invitrogen™) and transfected with vector pTT5 expressing His-VEGF-Ax^{Ala} using PEI (Polysciences™); the canonical TGA stop codon was mutated to GCA to ensure robust synthesis of VEGF-Ax-like protein. His-VEGF-Ax^{Ala} was purified from conditioned medium collected 6 days after transfection using HisTrap FF crude column (GE healthcare™). Purified His-VEGF-Ax^{Ala} was detected with anti-VEGF-Ax, anti-VEGF-Ab and anti-VEGFA antibodies.

Fluorescence microscopy. All tissue arrays contained 1.5-mm by 5-μm thick samples (US Biomax™). The adenocarcinoma array had samples from 30 cases and from 5 healthy individuals. Sections were deparaffinized, rehydrated, and subjected to antigen retrieval using Target retrieval solution (Dako™) at 95° C. for 25 min. Sections were blocked with 5% horse serum for 2 h, incubated with anti-VEGF-A or -VEGF-Ax antibodies or IgG overnight at 4° C., and then stained with Alexa Fluor 488-conjugated secondary antibody at room temperature for 1 h. Fluorescence intensity was quantified using NIH ImageJ software, and background fluorescence from IgG samples was subtracted.

Frozen human tissue sections were fixed with 4% paraformaldehyde in PBS, permeabilized with 0.1% Triton X-100, and incubated with anti-VEGF-Ax antibody followed by Alexa Fluor 488-conjugated secondary antibody. The same protocol was used to image hnRNP A2/B1 in cultured ECs. Single-plane, confocal images were obtained with a 1.0 Airy pinhole (Leica DMRXE with confocal TCS SP2 unit).

Endothelial cell tube formation. Bovine aortic ECs were pretreated with 50 ng/ml of His-VEGF-Ax^{Ala} for 6 h after which they were (10⁵ cells/cm²) seeded on growth factor-reduced LDEV-free Matrigel (BD Biosciences™) placed on μ-slide developed for angiogenesis assays (Ibidi). Incubation with His-VEGF-Ax^{Ala} was continued for 10 h. Cells were then fixed with 4% paraformaldehyde, stained with CyQUANT dye and imaged. Total tube length was quantified using ImageJ.

Genome-wide analysis of readthrough. 3′UTRs from H. sapiens, M. mulatta, B. taurus, M. musculus, and R. norvegicus were retrieved from the UTR database. 3′UTR sequences from 6357 genes common to the five species were translated in silico using BioPerl translate module. First 60-amino acid sequence of each 3′UTR were aligned and scored. Using a cut-off of 1400, heuristically chosen to include scores >70% of VEGFA score, 539 genes were selected for manual screening; mRNAs that undergo alternative splicing at the 3′UTR, lack downstream, in-frame stop codons, or exhibit conservation after the downstream stop codon were eliminated.

Statistical tests: Unless mentioned otherwise Student's t-test was employed to test the significance of differences we observed in various experiments. Mann-Whitney test was used for the analysis shown in FIG. 10H.

The complete disclosure of all patents, patent applications, and publications, and electronically available material cited herein are incorporated by reference. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

What is claimed is:

1. An isolated VEGF-Ax polypeptide having anti-angiogenic activity, the polypeptide comprising a region having at least 85% identity to SEQ ID NO: 1 and having an amino terminus comprising a region having at least 85% identity to SEQ ID NO: 2.

2. The isolated VEGF-Ax polypeptide of claim 1, wherein the polypeptide has at least 85% identity to SEQ ID NO: 4 (VEGF-A121x).

3. The isolated VEGF-Ax polypeptide of claim 1, wherein the polypeptide has at least 85% identity to SEQ ID NO: 6 (VEGF-A165x).

4. A method of treating or preventing angiogenesis by administering to a subject a therapeutically effective amount of a VEGF-Ax polypeptide comprising a region having at least 85% identity to SEQ ID NO: 1 and having an amino terminus comprising a region having at least 85% identity to SEQ ID NO: 2.

5. The method of claim 4, wherein the VEGF-Ax polypeptide has at least 85% identity to SEQ ID NO: 4 (VEGF-A121x).

6. The method of claim 4, wherein the VEGF-Ax polypeptide has at least 85% identity to SEQ ID NO: 6 (VEGF-A165x).

7. The method of claim 4, wherein the subject has been diagnosed as having cancer.

8. The method of claim 4, wherein the VEGF-Ax polypeptide is administered in a pharmaceutically acceptable carrier.

9. An isolated Ax polypeptide having anti-angiogenic activity, the polypeptide having from 22 to 50 amino acids and comprising a region having at least 85% identity to SEQ ID NO: 2.

10. The isolated Ax polypeptide of claim 9, wherein the polypeptide has from 22 to 30 amino acids.

11. A method of decreasing angiogenesis by administering to a subject a therapeutically effective amount of an Ax polypeptide having from 22 to 50 amino acids and comprising a region having at least 85% identity to SEQ ID NO: 2.

12. The method of claim 11, wherein the Ax polypeptide has from 22 to 30 amino acids.

13. The method of claim 11, wherein the subject has been previously diagnosed as having cancer.

14. The method of claim 11, wherein the Ax polypeptide is administered in a pharmaceutically acceptable carrier.

15. An antibody specific to the Ax region of a VEGF-Ax polypeptide capable of binding to a VEGF-Ax and not capable of binding to VEGF-A.

16. The antibody of claim 15, wherein antibody is specific for a portion of the peptide having at least 85% sequence identity to SEQ ID NO: 9.

17. The antibody of claim 15, wherein the antibody is a monoclonal antibody.

18. The antibody of claim 15, wherein the antibody is a polyclonal antibody.

19. A method of prognosis of a disease or disorder associated with increased or abnormal angiogenesis in a subject, comprising the steps of:
a) determining the level of VEGF-Ax in a biological sample from the subject by an immunoassay using an antibody specific to the Ax region of a VEGF-Ax polypeptide that is capable of binding to a VEGF-Ax and not capable of binding to VEGF-A;
b) comparing the level of VEGF-Ax to a predetermined value based on levels of VEGF-Ax in comparable biological samples obtained from one or more control subjects;
c) providing a prognosis of increased severity of the disease or disorder for the subject if the level of VEGF-Ax in the subject is lower than the predetermined value.

20. The method of claim 19, wherein the disease is cancer.

21. The method of claim 19, wherein the biological sample is a blood, plasma, or serum sample.

22. The method of claim 19, wherein the immunoassay is an ELISA.

23. A method of stimulating angiogenesis in a subject, comprising administering a therapeutically effective amount of an antibody specific to the Ax region of a VEGF-Ax polypeptide that is capable of binding to VEGF-Ax and not capable of binding to VEGF-A.

24. The method of claim 23, wherein the antibody is a monoclonal antibody.

25. The method of claim 23, wherein the antibody is a polyclonal antibody.

26. The method of claim 23, wherein the subject has coronary artery disease.

27. An antibody specific to the C-terminus region of a VEGF-A polypeptide that is capable of binding to the C-terminus region of a VEGF-A polypeptide and not capable of binding to VEGF-Ax.

28. The antibody of claim 27, wherein the C-terminus region has the amino acid sequence SEQ ID NO: 10.

29. The antibody of claim 27, wherein the antibody is a monoclonal antibody.

30. The antibody of claim 27, wherein the antibody is a polyclonal antibody.

31. A method of decreasing angiogenesis in a subject, comprising administering a therapeutically effective amount of an antibody specific to the C-terminus region of a VEGF-A polypeptide that is capable of binding to the C-terminus region of a VEGF-A polypeptide and not capable of binding to VEGF-Ax.

32. The method of claim 31, wherein the subject has been previously diagnosed as having cancer.

33. An isolated Ax polypeptide fragment having anti-angiogenic activity, the polypeptide having from 5 to 20 amino acids and an amino acid sequence selected from within SEQ ID NO: 1.

34. The isolated Ax polypeptide fragment of claim 33, wherein the polypeptide has 6 amino acids.


SEQUENCE LISTING (45 sequences)

SEQ ID NO: 1 - protein, 22 aa, Unknown
  Description of Unknown VEGF-Ax peptide
SAGLEEGASLRVSGTRSLTRKD

SEQ ID NO: 2 - protein, 110 aa, Unknown
  Description of Unknown Exons 1-5 of VEGF-A polypeptide
MNFLLSWVHWSLALLLYLHHAKWSQAAPMAEGGQKPHEVVKFMDVYQRSFCRPIETLVDIFQEYPDEIEFIFKPSCVPLMRCGGCCNDESLECVPTEEFNITMQIMRIKP

SEQ ID NO: 3 - protein, 133 aa, Unknown
  Description of Unknown VEGF-Ax isoform VEGF-A111x polypeptide
APMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRCDKPRRSAGLEEGASLRVSGTRSLTRKD

SEQ ID NO: 4 - protein, 143 aa, Unknown
  Description of Unknown VEGF-Ax isoform VEGF-A121x polypeptide
APMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQEKCDKPRRSAGLEEGASLRVSGTRSLTRKD

SEQ ID NO: 5 - protein, 170 aa, Unknown
  Description of Unknown VEGF-Ax isoform VEGF-A148x polypeptide
APMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQENPCGPCSERRKHLFVQDPQTCKCSCKNTDSRCKMSAGLEEGASLRVSGTRSLTRKD

SEQ ID NO: 6 - protein, 187 aa, Unknown
  Description of Unknown VEGF-Ax isoform VEGF-A165x polypeptide
APMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQENPCGPCSERRKHLFVQDPQTCKCSCKNTDSRCKARQLELNERTCRCDKPRRSAGLEEGASLRVSGTRSLTRKD

SEQ ID NO: 7 - protein, 205 aa, Unknown
  Description of Unknown VEGF-Ax isoform VEGF-A183x polypeptide
APMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQEKKSVRGKGKGQKRKRKKSRPCGPCSERRKHLFVQDPQTCKCSCKNTDSRCKARQLELNERTCRCDKPRRSAGLEEGASLRVSGTRSLTRKD

SEQ ID NO: 8 - protein, 211 aa, Unknown
  Description of Unknown VEGF-Ax isoform VEGF-A189x polypeptide
APMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQEKKSVRGKGKGQKRKRKKSRYKSWSVPCGPCSERRKHLFVQDPQTCKCSCKNTDSRCKARQLELNERTCRCDKPRRSAGLEEGASLRVSGTRSLTRKD

SEQ ID NO: 9 - protein, 16 aa, Artificial Sequence
  Description of Artificial Sequence Synthetic peptide
SAGLEEGASLRVSGTR

SEQ ID NO: 10 - protein, 6 aa, Unknown
  Description of Unknown VEGF-A pro-angiogenic peptide
CDKPRR

SEQ ID NO: 11 - protein, 6 aa, Unknown
  Description of Unknown VEGF-A anti-angiogenic peptide
SLTRKD

SEQ ID NO: 12 - protein, 7 aa, Unknown
  Description of Unknown VEGF-A peptide
CDKPRRS

SEQ ID NO: 13 - protein, 7 aa, Unknown
  Description of Unknown VEGF-A peptide
CKKPRRS

SEQ ID NO: 14 - protein, 6 aa, Unknown
  Description of Unknown VEGF-A peptide
XLTRKD

SEQ ID NO: 15 - protein, 12 aa, Unknown
  Description of Unknown VEGF-A peptide
RSAGLEEGASLR

SEQ ID NO: 16 - DNA, 11 bp, Artificial Sequence
  Description of Artificial Sequence Synthetic oligonucleotide
gccaaggagcc

SEQ ID NO: 17 - DNA, 25 bp, Artificial Sequence
  Description of Artificial Sequence Synthetic oligonucleotide
acgtctggttcccgaaaccctgagg

SEQ ID NO: 18 - DNA, 25 bp, Unknown
  Description of Unknown Bovine VEGF-A target oligonucleotide
gcttcctacagcataacaaatgtga

SEQ ID NO: 19 - DNA, 19 bp, Unknown
  Description of Unknown Bovine VEGF-A target oligonucleotide
ggagtacccagatgagatt

SEQ ID NO: 20 - DNA, 19 bp, Unknown
  Description of Unknown Bovine VEGF-A target oligonucleotide
atgtgaatgcagaccaaag

SEQ ID NO: 21 - DNA, 25 bp, Unknown
  Description of Unknown Bovine VEGF-A target oligonucleotide
ggctttgtctagacaagaaatgcag

SEQ ID NO: 22 - DNA, 32 bp, Artificial Sequence
  Description of Artificial Sequence Synthetic primer
atgcaagcttatgaactttctgctctcttggg

SEQ ID NO: 23 - DNA, 31 bp, Artificial Sequence
  Description of Artificial Sequence Synthetic primer
atgcggatccgtctttcctggtgagacgtct

SEQ ID NO: 24 - DNA, 19 bp, Artificial Sequence
  Description of Artificial Sequence Synthetic primer
taatacgactcactatagg

SEQ ID NO: 25 - DNA, 19 bp, Artificial Sequence
  Description of Artificial Sequence Synthetic primer
atgcggatcaaacctcacc

SEQ ID NO: 26 - DNA, 18 bp, Artificial Sequence
  Description of Artificial Sequence Synthetic primer
gtctttcctggtgagacg

SEQ ID NO: 27 - DNA, 30 bp, Artificial Sequence
  Description of Artificial Sequence Synthetic oligonucleotide
ggcggctccggcggctccctcgtgctcgag

SEQ ID NO: 28 - RNA, 44 nt, Artificial Sequence
  Description of Artificial Sequence Synthetic oligonucleotide
ugugacaagccgaggcggugagccgggcaggaggaaggagccuc

SEQ ID NO: 29 - RNA, 72 nt, Homo sapiens
cggugagccgggcaggaggaaggagccucccucaggguuucgggaaccagaucucucaccaggaaagacuga

SEQ ID NO: 30 - RNA, 72 nt, Nomascus leucogenys; identical to SEQ ID NO: 29

SEQ ID NO: 31 - RNA, 72 nt, Pongo abelii; identical to SEQ ID NO: 29

SEQ ID NO: 32 - RNA, 72 nt, Macaca fascicularis; identical to SEQ ID NO: 29

SEQ ID NO: 33 - RNA, 72 nt, Callithrix jacchus
cggugagccgggcaggaggacggagccucccucaggguuucgggaaccagaucucucaccaggaaagacuga

SEQ ID NO: 34 - RNA, 72 nt, Ailuropoda melanoleuca
cggugagccgggcuggaagaaggagccucccucaggguuucgggaaccagaccucucaccaggaaagccuga

SEQ ID NO: 35 - RNA, 72 nt, Bos taurus
cggugagccgggcuggaggaaggagccucccucaggguuucgggaaccagacgucucaccaggaaagacuga

SEQ ID NO: 36 - RNA, 72 nt, Ovis aries; identical to SEQ ID NO: 35

SEQ ID NO: 37 - RNA, 72 nt, Capra hircus; identical to SEQ ID NO: 35

SEQ ID NO: 38 - RNA, 72 nt, Canis familiaris; identical to SEQ ID NO: 34

SEQ ID NO: 39 - RNA, 75 nt, Rattus norvegicus
cggugagccaggcugcaggaaggagccucccucaggguuucgggaacuagaccucucaccggaaagaccgauuaa

SEQ ID NO: 40 - RNA, 75 nt, Mus musculus
cggugagccaggcugcaggaaggagccucccucaggguuucgggaaccagaccucucaccggaaagaccgauuaa

SEQ ID NO: 41 - DNA, 69 bp, Unknown
  Description of Unknown Bovine Ax element oligonucleotide
tgagccgggctggaggaaggagcctccctcagggtttcgggaaccagacgtctcaccaggaaagactga

SEQ ID NO: 42 - DNA, 84 bp, Artificial Sequence
  Description of Artificial Sequence Synthetic oligonucleotide
gacaagccgaggcggugagccgggcaggaggaaggagccucccucaggguuucgggaaccagatctctcaccaggaaagacuga

SEQ ID NO: 43 - RNA, 12 nt, Artificial Sequence
  Description of Artificial Sequence Synthetic oligonucleotide
gccaaggagccc

SEQ ID NO: 44 - DNA, 11 bp, Artificial Sequence
  Description of Artificial Sequence Synthetic oligonucleotide
aggaaggagcc

SEQ ID NO: 45 - DNA, 11 bp, Artificial Sequence
  Description of Artificial Sequence Synthetic oligonucleotide
aggttggagcc